(19) United States
(12) Patent Application Publication
(10) Pub. No.: US 2005/0032869 A1
(43) Pub. Date: Feb. 10, 2005

(54) Pyrazolyl-indole derivatives active as kinase inhibitors, process for their preparation and pharmaceutical compositions comprising them

(75) Inventors: Daniela Gavina Berta, Sassari (IT); Barbara Forte, Milano (IT); Sergio Mantegani, Milano (IT); Mario Varasi, Milano (IT); Paola Vianello, Milano (IT)

Correspondence Address: Peter I. Bernstein;Scully, Scott, Murphy & Presser, 400 Garden City Plaza, Garden City, NY, 11530, US

(73) Assignee: Pharmacia Italia S.p.A., Milan (IT)

(21) Appl. No.: 10/882,492

(22) Filed: Jul. 1, 2004

Related U.S. Application Data
(60) Provisional application No. 60/485,406, filed on Jul. 8, 2003.

(51) Int. Cl. A61K 31/416

(52) U.S. Cl. 514/406

(57) ABSTRACT

Pyrazolyl-indole derivatives of formula (I) as defined in the specification, and pharmaceutically acceptable salts thereof, process for their preparation and pharmaceutical compositions comprising them are disclosed; the compounds of the invention may be useful, in therapy, in the treatment of diseases associated with a disregulated protein kinase activity, like cancer.

### CROSS REFERENCE TO RELATED APPLICATIONS

The present application claims benefit of U.S. Provisional Application No. 60/485,406 filed Jul. 8, 2003.

### BACKGROUND OF THE INVENTION

1. Field of the Invention

The present invention relates to indole derivatives and, more in particular, to pyrazolyl-indole derivatives active as kinase inhibitors, to a process for their preparation, to pharmaceutical compositions comprising them and to their use as therapeutic agents, particularly in the treatment of diseases linked to disregulated protein kinases.

2. Discussion of the Background

The malfunctioning of protein kinases (PKs) is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

### SUMMARY OF THE INVENTION

It is an object of the invention to provide compounds which are useful in therapy as agents against a host of diseases caused by and/or associated to a disregulated protein kinase activity.

It is another object to provide compounds which are endowed with protein kinase inhibiting activity.

The present inventors have now discovered that some pyrazolyl-indoles, and derivatives thereof, are endowed with protein kinase inhibiting activity and are thus useful in therapy in the treatment of diseases associated with disregulated protein kinases.

More specifically, the compounds of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma.

Due to the key role of PKs in the regulation of cellular proliferation, these pyrazolyl-indoles are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

The compounds of the invention can be useful in the treatment of Alzheimer's disease, as suggested by the fact that cdk5 is involved in the phosphorylation of tau protein (J. Biochem., 117, 741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis, as well as in the treatment of organ transplant rejection and host versus graft disease.

The compounds of the invention may also act as inhibitor of other protein kinases, e.g., cyclin-dependent kinases (cdk) such as cdk2 and cdk5, protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, PI3K, weel kinase, Src, Abl, Akt, MAPK, ILK, MK-2, IKK-2, Cdc7, Nek, and thus be effective in the treatment of diseases associated with other protein kinases.

The compounds of the invention are also useful in the treatment and prevention of radiotherapy-induced or chemotherapy-induced alopecia.

Several heterocyclic compounds are known in the art as therapeutic agents or even as protein kinase inhibitors.

Among them are some pyrazole derivatives disclosed in the international patent applications WO 01/12189, WO 01/12188, WO 02/48114, WO 02/070515, WO 99/32455 and WO 02/062804, all in the name of the Applicant itself and herewith incorporated by reference.

Indole derivatives further substituted by indazolyl groups have been also disclosed as protein kinase inhibitors in WO 01/53268 and WO 01/02369; benzodiazepine derivatives substituted by indolyl moieties and possessing cdk2 inhibitory activity have been disclosed in WO 00/64900; pyrazolone derivatives possessing protein kinase inhibitory activity have been disclosed in WO 01/32653; pyrazolyl-indoles substituted by propenone groups in position 3 of the indole moiety have been disclosed as antitumor agents in WO 95/14003.

Indole derivatives among which are indolyl-indazoles as possessing tyrosine kinase inhibitory activity are also disclosed in WO 03/024969.

Benzimidazole derivatives endowed with KDR protein kinase inhibitory activity are disclosed in WO 03/035644.

In addition to the above, general formula compounds comprising pyrazole derivatives are known in the art as antitumor, antimicrobial or fungicide agents, as well as for the treatment and prophylaxis of anaemias or as factor Xa inhibitors, for instance as disclosed in WO 97/28158, WO 00/39108, WO 00/46207, WO 00/46208, WO 01/82930 and in Chemical Abstracts C.A.88(1978):31980.

Accordingly, the present invention provides a method for treating diseases caused by and/or associated with an altered protein kinase activity, by administering to a mammal in need thereof an effective amount of a compound of formula (I) 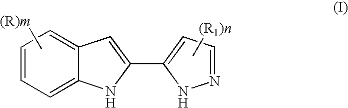
wherein
R is hydrogen, halogen, nitro, cyano, hydroxy, or it is a group optionally further substituted selected from straight or branched C₁-C₆ alkyl or C₁-C₆ alkoxy, C₃-C₆ cycloalkyl, aryl, heterocyclyl, or it is a group —NR′R″, —CONR′R″, —NR′COR″, —COOR′ or —SO₂NR′R″, wherein R′ and R″ are, the same or different and independently in each occasion, a hydrogen atom or a group optionally further substituted selected from straight or branched C₁-C₆ alkyl, C₃-C₆ cycloalkyl, aryl or heterocyclyl; or, taken together with the nitrogen atom to which they are attached, R′ and R″ may form a 5 or 6 membered nitrogen containing heterocycle, optionally comprising one additional heteroatom selected among N, O or S;
R₁ has the meanings above reported to R but other than hydroxy;
m is an integer from 1 to 4;
n is 1 or 2; and the pharmaceutically acceptable salts thereof.

In a preferred embodiment of the method described above, the disease caused by and/or associated with an altered protein kinase activity is selected from the group consisting of cancer, cell proliferative disorders, Alzheimer's disease, viral infections, autoimmune diseases and neurodegenerative disorders.

Specific types of cancer that may be treated include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

The present invention further provides a compound of formula (I) 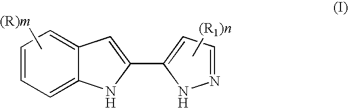
wherein
R is hydrogen, halogen, nitro, cyano, hydroxy, or it is a group optionally further substituted selected from straight or branched C₁-C₆ alkyl or C₁-C₆ alkoxy, C₃-C₆ cycloalkyl, aryl, heterocyclyl, or it is a group —NR′R″, —CONR′R″, —NR′COR″, —COOR′ or —SO₂NR′R″, wherein R′ and R″ are, the same or different and independently in each occasion, a hydrogen atom or a group optionally further substituted selected from straight or branched C₁-C₆ alkyl, C₃-C₆ cycloalkyl, aryl or heterocyclyl; or, taken together with the nitrogen atom to which they are attached, R′ and R″ may form a 5 or 6 membered nitrogen containing heterocycle, optionally comprising one additional heteroatom selected among N, O or S;
R₁ has the meanings above reported to R but other than hydroxy;
m is an integer from 1 to 4;
n is 1 or 2;
and the pharmaceutically acceptable salts thereof.

### DETAILED DESCRIPTION OF THE INVENTION

The compounds of formula (I), object of the present invention, may have asymmetric carbon atoms and may therefore exist either as racemic admixtures or as individual optical isomers. Accordingly, all the possible isomers and their admixtures and of both the metabolites and the pharmaceutically acceptable bio-precursors (otherwise referred to as pro-drugs) of the compounds of formula (I), as well as any therapeutic method of treatment comprising them, are also within the scope of the present invention.

In the present description, unless otherwise indicated, with the term halogen atom we intend a fluorine, chlorine, bromine or iodine atom.

With the term straight or branched C₁-C₆ alkyl or alkoxy we intend a group such as, for instance, methyl, ethyl, n-propyl, isopropyl, n-butyl, isobutyl, sec-butyl, tert-butyl, n-pentyl, n-hexyl, methoxy, ethoxy, n-propoxy, isopropoxy, n-butoxy, isobutoxy, sec-butoxy, tert-butoxy, n-pentyloxy, n-hexyloxy, and the like.

With the term C₃-C₆ cycloalkyl we intend a group such as cyclopropyl, cyclobutyl, cyclopentyl or cyclohexyl.

With the term aryl we intend a mono- or bi- either carbocyclic as well as heterocyclic hydrocarbon with from 1 to 2 ring moieties, either fused or linked to each other by single bonds, wherein at least one of the carbocyclic or heterocyclic rings is aromatic.

Non limiting examples of aryl groups are, for instance, phenyl, indanyl, biphenyl, α- or β-naphthyl, pyridyl, pyrazinyl, pyrimidinyl, pyridazinyl, 1,3,5-triazinyl, indolyl, imidazolyl, inmidazopyridyl, 1,2-methylenedioxyphenyl, thiazolyl, isothiazolyl, pyrrolyl, pyrrolyl-phenyl, furyl, phenyl-furyl, benzotetrahydrofuranyl, oxazolyl, isoxazolyl, pyrazolyl, chromenyl, thienyl, benzothienyl, isoindolinyl, benzoimidazolyl, benzoxazolyl, benzothiazolyl, isoindolinyl-phenyl, quinolinyl, isoquinolinyl, quinoxalinyl, pyrazinyl, benzofurazanyl, 1,2,3-triazolyl, 1-phenyl-1,2,3-triazolyl, and the like.

With the term heterocycle or heterocyclyl we intend a 5 or 6 membered heterocycle, hence encompassing aromatic heterocyclic groups also referred to as heteroaryl groups and being comprised within the meanings of aryl. In addition, with the term heterocyclyl we further intend a saturated or partially unsaturated 5 or 6 membered carbocycle wherein one or more carbon atoms are replaced by 1 to 3 heteroatoms or heteroatomic groups such as N, NR′, O or S, wherein R′ is as defined in the general formula.

Additional examples of 5 or 6 membered heterocyclyl groups optionally benzocondensed or further substituted, besides those previously referred to as aryl groups, are 1,3-dioxolane, pyran, pyrrolidine, pyrroline, imidazolidine, pyrazolidine, pyrazoline, piperidine, piperazine, morpholine, tetrahydrofuran, and the like.

Moreover, R may also represent a functional group linked to the benzene moiety of the compound of formula (I), being selected from amino (—NR′R″), amido (—CONR′R″ or —NR′COR″), sulfonamido (—SO₂NR′R″) or carboxy (—COOR′), wherein R′ and R″ are as above defined.

According to this latter aspect, it is clear to the skilled person that in the case of R (or R₁) being defined as a group —NR′R″, —CONR′R″ or —SO₂NR′R″, R′ and R″ groups may be also combined together so as to form a 5 or 6 membered heterocyclic ring, at least containing the nitrogen atom to which R′ and R″ are bonded and, optionally, an additional heteroatom selected among N, Oor S.

Non limiting examples of the said heterocycles may thus comprise, for instance, pyrrole, pyrazole, imidazole, pyrrolidine, pyrroline, imidazolidine, imidazoline, pyrazolidine, pyrazoline, piperidine, piperazine, morpholine, and the like.

As set forth in the general formula, R₁ is a group linked to the pyrazole moiety of the compound of formula (I), having any one of the meanings provided to R other than hydroxy.

From all of the above, it is clear to the skilled person that the compounds of formula (I) may bear from 1 to 4 R groups in positions 4, 5, 6 and 7; and 1 or 2 R₁ groups in positions 3′ and 4′, according to the numbering system below:

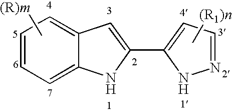

According to the above meanings provided to R, R₁, R′ and R″, any of the said groups may be further optionally substituted in any of the free positions by one or more groups, for instance 1 to 6 groups, selected from: halogen, nitro, oxo groups (═O), carboxy, cyano, alkyl, perfluorinated alkyl, hydroxyalkyl, alkenyl, alkynyl, cycloalkyl, aryl, heterocyclyl, amino groups and derivatives thereof such as, for instance, alkylamino, dialkylamino, cycloalkylamino, arylamino, diarylamino, arylalkylamino, ureido, alkylureido or arylureido; carbonylamino groups and derivatives thereof such as, for instance, formylamino, alkylcarbonylamino, alkenylcarbonylamino, arylcarbonylamino, alkoxycarbonylamino; hydroxy groups and derivatives thereof such as, for instance, alkoxy, aryloxy, heterocyclyloxy, alkylcarbonyloxy, arylcarbonyloxy, cycloalkenyloxy or alkylideneaminooxy; carbonyl groups and derivatives thereof such as, for instance, alkylcarbonyl, arylcarbonyl, alkoxycarbonyl, aryloxycarbonyl, cycloalkyloxycarbonyl, aminocarbonyl, alkylaminocarbonyl, dialkylaminocarbonyl; sulfurated derivatives such as, for instance, alkylthio, arylthio, alkylsulfonyl, arylsulfonyl, alkylsulfinyl, arylsulfinyl, arylsulfonyloxy, aminosulfonyl, alkylaminosulfonyl or dialkylaminosulfonyl. In their turn, whenever appropriate, each of the above groups may be further substituted by one or more of the aforementioned groups.

With the term perfluorinated alkyl we intend a straight or branched C₁-C₆ alkyl group as above defined, wherein all hydrogen atoms are replaced by fluorine atoms. Example of perfluorinated alkyl groups are, for instance, trifluoromethyl, 2,2,2-trifluoroethyl, 1,2-difluoroethyl, 1,1,1,3,3,3-hexafluoropropyl-2-yl and the like.

With the term alkenyl or alkynyl we intend a straight or branched unsaturated hydrocarbon chain with from 2 to 6 carbon atoms, having a double or triple bond such as, for instance, vinyl, ethynyl, 1-propenyl, allyl, 1- or 2-propynyl, 1-, 2- or 3-butenyl, 1-, 2- or 3-butynyl, pentenyl, pentynyl, hexenyl, hexynyl and the like.

From all of the above, it is clear to the skilled person that any group whose name has been identified as a composite name such as, for instance, cycloalkylalkyl, arylalkyl, heterocyclylalkyl, alkoxy, alkylthio, aryloxy, arylalkoxy, heterocyclyloxy, heterocyclylalkoxy, alkylcarbonyloxy and the like, has to be intended as conventionally construed from the parts to which they derive.

As an example, the term heterocyclyl-alkyl stands for an alkyl group being further substituted by a heterocyclyl group, wherein alkyl and heterocyclyl are as above defined.

Pharmaceutically acceptable salts of the compounds of formula (I) are the acid addition salts with inorganic or organic, e.g. nitric, hydrochloric, hydrobromic, sulfuric, perchloric, phosphoric, acetic, trifluoroacetic, propionic, glycolic, lactic, oxalic, malonic, malic, maleic, tartaric, citric, benzoic, cinnamic, mandelic, methanesulfonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

When referring to the compounds of formula (I) of the invention, it is also clear to the skilled person that the unsubstituted ring nitrogen pyrazole is known to rapidly equilibrate, in solution, as a mixture of tautomers of formula (Ia) and (lb) which are both comprised within the scope of the invention

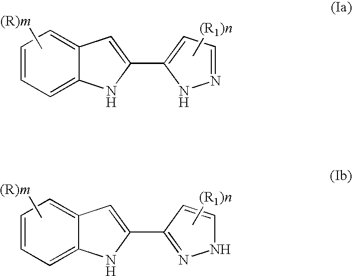

A first class of preferred compounds of the invention is represented by the derivatives of formula (I) wherein R is a hydrogen or halogen atom, R₁ is a hydrogen atom or a group selected from cyano, —COOR′ or —CONR′R″, wherein R′ and R″ have the above reported meanings, and m and n are both 1.

Another class of preferred compounds of the invention is represented by the derivatives of formula (I) wherein R is a group —COOR′ or —CONR′R″, wherein R′ and R″ have the above reported meanings, R₁ is hydrogen, and m and n are both 1.

For a general reference to any specific example of the compounds of formula (I) of the invention, whenever appropriate in the form of pharmaceutically acceptable salts, see the experimental section and claims.

As set forth above, it is a further object of the present invention a process for preparing the compounds of formula (I) which may be carried out either in solution, according to a classical synthetic approach or, alternatively, under solid-phase-synthesis (SPS) conditions.

These latter conditions are particularly advantageous when preparing libraries of compounds according to combinatorial chemistry techniques, for instance as reported below.

Therefore, the compounds of formula (I) and the pharmaceutically acceptable salts thereof may be prepared by a process comprising:
a) coupling, in the presence of a suitable catalyst, the compound of formula (II) with the compound of formula (III)× 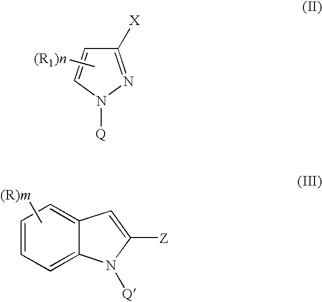
   wherein R, R₁, m and n are as above defined; Q and Q′, the same or different from each other, may represent suitable nitrogen protective groups or polymeric solid supports; X is a halogen atom or a group selected from methylsulfonyloxy, trifluoromethylsulfonyloxy, phenylsulfonyloxy or fluorido-sulphate (—OSO₂F); and Z is selected from halogen, boronic acid, boronate, trialkyl-stannane, trihalostannane, zinc halide, cuprate, alkyldihalo-sylane or a Grignard salt; so as to obtain a compound of formula (IV) 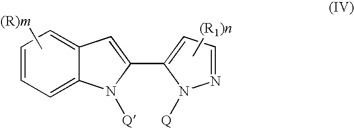
b) optionally converting the compound of formula (IV) into another compound of formula (IV); and
c) deprotecting or cleaving from the resin Q and Q′ the compound of formula (IV), so as to obtain the compound of formula (I) and, whenever desired, converting it into a pharmaceutically acceptable salt thereof.

According to step (a) of the process, the reaction between the compounds of formula (II) and (III) is carried out in the presence of a suitable catalyst such as, for instance, tetrakis(triphenylphosphine)palladium, tris(dibenzylideneacetone)dipalladium, palladium chloride, bis(triphenylphosphine)palladium chloride, palladium acetate, nickel chloride, 1,2-bis (diphenylphosphino) ethane nickel chloride, dichlorobis(tributylphosphine)nickel, nickel acetylacetonate and of a suitable ligand such as triphenylphosphine, tri-2-furylphosphine, tributyiphosphine, 2-dicyclohexylphosphino-2′-(N,N-dimethylamino)biphenyl, triphenylarsine.

The reaction is carried out under basic conditions, for instance in the presence of sodium carbonate, potassium carbonate, cesium carbonate, thallium carbonate, sodium hydroxide, barium hydroxide, triethylamine or diisopropylethylamine, in a suitable solvent such as dimethoxyethane, tetrahydrofuran, ethanol, water, toluene, ethanol or 4-dioxane, at a temperature ranging from room temperature to refluxing temperature, for a suitable time varying from about 30 minutes to about 96 hours.

Preferably, this reaction is carried out with tetrakis(triphenylphosphine)palladium as the catalyst, and tallium carbonate as the base.

According to a preferred embodiment, within the compounds of formula (II) and (III), X is a iodine atom and Z is a boronic acid [-B(OH)₂] or tributyl stannane.

As far as Q and Q′ are concerned, they may represent a suitable nitrogen protecting group such as, for instance, trityl, trimethylsilylethoxymethyl (SEM), tert-butoxycarbonyl (boc), ethylcarbamate or trichloroethylcarbamate. Alternatively, one or both of Q and Q′ may also represent a suitable inert polymeric resin otherwise defined as polymeric solid support such as, for instance, trityl resin, chloro-trityl resin, methylisocyanate resin, p-nitrophenyl carbonate Wang resin, isocyanate polystyrenic resin or the like, which are all conventionally known in this field.

Preferably, Q represents a trimethylsilylethoxymethyl or ethylcarbamate group or it is a trityl resin and Q′ is tert-butoxycarbonyl or trimethylsilylethoxymethyl.

For a general reference on aryl-aryl cross coupling reactions, as per step (a) of the process, see Miyaura, Norio et al., Palladium-Catalyzed Cross-Coupling Reactions of Organoboron Compounds [Chemical Reviews (1995), 95(7), 245783]; and Hassan, Jwanro et al., Aryl-Aryl Bond Formation One Century after the Discovery of the Ullmann Reaction [Chemical Reviews (2002), 102(5), 1359-1469].

The compounds of formula (IV) thus obtained may be then converted in a variety of ways, according to step (b) of the process, into other compounds of formula (IV), by working according to conventional methods.

As an example, the compounds of formula (IV) wherein any one of R and R₁ is a group —COOR′ wherein R′ is as above defined, may be converted into the corresponding derivatives of formula (IV) wherein R′ is hydrogen. The above reaction is carried out according to conventional methods which enable, for instance, hydrolysis of carboxy ester groups, e.g. under basic conditions in the presence of suitable bases such as sodium, potassium or lithium hydroxide, and in a suitable solvent such as N,N-dimethylformamide, methanol, ethanol, tetrahydrofuran, water, and mixtures thereof. Typically, the reaction is carried out at temperatures ranging from room temperature to refluxing temperature and for a time varying from about 30 minutes to about 96 hours.

Likewise, the compounds of formula (IV) thus obtained and wherein any one of R and R₁ is a group —COOH may be then converted into a variety of derivatives bearing the corresponding —CONR′R″ group wherein R′ and R″ are as above defined. Also this reaction is carried out according to well known methods for preparing carboxamides and may comprise, for instance, the reaction of the above carboxylic acid derivative with a suitable amine HNR′R″.

Typically, this reaction is carried out in the presence of a coupling agent such as, for instance, benzotriazol-1-yloxytris(pyrrolidino)phosphonium-hexafluorophosphate-carbodiimide, 1,3-dicyclohexylcarbodiimide, bromo-tris-pyrrolidino-phosphonium hexafluorophosphate, 1,3-diisopropylcarbodiimide, o-benzotriazol-1-yl-N,N,N′,N′-tetramethyluronium tetrafluoroborate, 1-(3-dimethylaminopropyl)-3-ethylcarbodiimide, N-cyclohexylcarbodiimide-N′-propyloxymethyl polystyrene or N-cyclohexylcarbodiimide-N′-methyl polystyrene, in a suitable solvent such as, for instance, dichloromethane, chloroform, tetrahydrofuran, diethyl ether, 1,4-dioxane, acetonitrile, toluene or N,N-dimethylformamide, at a temperature ranging from about −10° C. to refluxing temperature and for a time varying from about 30 minutes to about 96 hours. The said reaction is optionally carried out in the presence of a suitable catalyst, for instance 4-dimethylaminopyridine or, alternatively, in the presence of a further coupling reagent such as N-hydroxybenzotriazole.

Alternatively, the above carboxamide preparation may be also accomplished through a mixed anhydride method, that is by using an alkyl chloroformate such as ethyl, iso-butyl or isopropyl chloroformate, in the presence of a tertiary base such as triethylamine, N,N-diisopropylethylamine or pyridine, in a suitable solvent such as, for instance, toluene, dichloromethane, chloroform, tetrahydrofuran, acetonitrile, diethyl ether, 1,4-dioxane, or N,N-dimethylformamide, and at a temperature ranging from about −30° C. to room temperature.

From all of the above, it is also clear to the skilled person that any group R or R₁, as well as any one of the optional substituents which are part of R, R₁, R′ or R″ and which are further susceptible of being converted into other groups may also lead to a variety of derivatives.

As a non limiting example, carboxy groups may be converted into a variety of derivatives including esters and amides; carboxamides may undergo reductive amination to amino derivatives; amines may be further acylated in a variety of ways to other carboxamides; alkylthio groups may be oxidized to alkylsulfonyl groups or even replaced by amino or alkoxy groups and derivatives thereof; nitro groups can be reduced to amines; and the like.

For a general reference to any one of the above reactions and which have been here conveniently grouped into step (b) of the process, see the experimental section.

According to step (c) of the process, the compound of formula (IV) is then deprotected from the Q and Q′ groups.

The above reaction is widely known in the art and is accomplished under acidic or basic conditions, depending upon the nature of the Q and Q′ groups themselves.

As an example of deprotection under acidic conditions, the compound of formula (IV) being obtained in step (b) may be treated with hydrochloric or trifluoroacetic acid.

Preferably, for instance in the case Q is trimethylsilylethoxymethyl, or trityl resin and Q′ is tert-butoxycarbonyl or trimethylsilylethoxymethyl, the reaction occurs by using a solution of hydrochloric acid at a concentration ranging from 0.5 to 3N in methanol, at a temperature varying from about 0° C. to refluxing temperature, and for a time of about 5 minutes to about 2 hours.

In step (c), deprotection or resin cleavage may be also carried out, depending upon the nature of the Q and Q′ groups, under basic conditions.

As an example, the reaction may be carried out in the presence of aqueous potassium or sodium hydroxide and in the presence of a suitable co-solvent such as methanol, ethanol, N,N-dimethylformamide, 1,4-dioxane or acetonitrile, so as to yield the desired compound of formula (I). The compound of formula (IV) may be thus suspended in a solution of 35% of sodium or potassium hydroxide, for instance in methanol, by working under mild operative conditions, for instance at temperatures ranging from about 5° C. to about 60° C. and for a time varying from about 2 hours to about a few days.

The starting material of formula (II) of the process is known or can be easily obtained according to known methods, for instance as per following scheme (a):

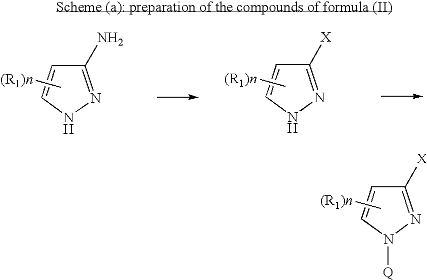

A suitable amino-pyrazole derivative may thus undergo diazotation reaction according to known methods by means of sodium nitrite, tert-butyl nitrite or, preferably, isoamyinitrite; the diazonium salt is then replaced by a suitable X group through reaction with a proper copper(I) halide such as, for instance, CuCl, CuBr, CuI, a trimethylsylyl chloride, bromide, iodide or even with iodine itself. Preferably, the reaction is carried out with isoamyl nitrite and diiodomethane at a temperature ranging from about 0° C. to about 150° C. and for a time varying from about 5 minutes to about 24 hours.

Subsequent protection at the pyrazole nitrogen atom or loading onto a solid support so as to yield the compound of formula (II) is also carried out according to known methods.

As an example, the intermediate pyrazole (—NH—) compound may be protected as trimethylsilylethoxymethyl (—NQ—) by reacting it with 2-(trimethylsilyl)ethoxymethyl chloride in the presence of sodium hydride, in a suitable solvent such as, for instance, tetrahydrofuran, diethyl ether, 1,4-dioxane, dichloromethane, chloroform, or N,N-dimethylformamide, at a temperature ranging from about 0° C. to room temperature and for a suitable time varying from about 30 minutes to about 96 hours.

Also the compounds of formula (III) are known or may be easily prepared according to known methods (see, for example, J. Chem. Soc. Perkin Trans., 1. 2000; 11, 1705-14), for instance as set forth in scheme (b) below:

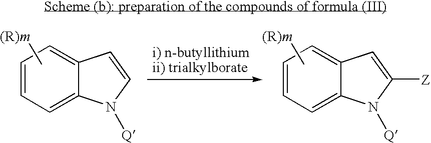

The nitrogen protected or otherwise polymer supported indole derivative, is prepared according to known methods, essentially as above reported in scheme (a) for the pyrazole intermediate.

The subsequent functionalization to yield the compound of formula (III) is then carried out in the presence of a base such as, for instance, n-butyllithium, sec-butyllithium, tert-butyllithium, lithium diisopropylamide, lithium 2,2,6,6-tetramethylpiperidide or a metal such as magnesium or litium, followed by quenching with a suitable trialkylborate, dioxaborolane, halotrialkyltin, zinc chloride, alkyl trihalosilane. The reaction is carried out in a suitable solvent like tetrahydrofuran, diethylether, dioxane or dimethoxyethane, at a temperature ranging from about 0° C. to 40° C. and for a suitable time varying from about 5 minutes to about 2 hours.

Preferably, the base is either n-butyllithium or lithium 2,2,6,6-tetramethylpiperidide. When the Z group is a boronic acid [-B(OH)₂], quenching is followed by hydrolysis with hydrochloric acid. In addition to the above, any protecting group, polymeric resin or any other reactant of the process of the invention, in any variant thereof, is known or can be prepared according to known methods.

According to an alternative approach, the compounds of formula (I) of the invention may be also prepared as per the synthetic pathway below, which represents a further object of the invention.

Therefore, the compounds of formula (I) and the pharmaceutically acceptable salts thereof may be prepared by a process comprising:
d) reacting a hydrazine derivative of formula (V) with a pyrazole derivative of formula (VI) 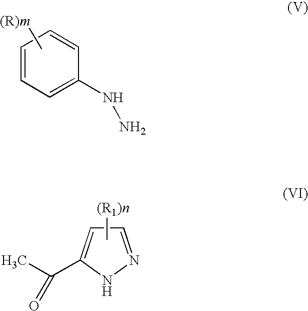
   wherein R, R₁, m and n are as above defined, so as to obtain a compound of formula (VIl) 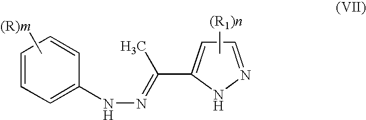
e) reacting the compound of formula (VII) under acidic conditions and in the presence of a Lewis acid, so as to obtain a compound of formula (I); and,
f) optionally converting it into another compound of formula (I) and/or into a pharmaceutically acceptable salt thereof.

According to step (d) of the process, the reaction between the compounds of formula (V) and (VI) is carried out in the presence of catalytic amounts of a suitable acid such as, for instance, hydrochloric, acetic, sulfuric or p-toluensulfonic acid, and in a solvent such as methanol, ethanol, benzene, toluene or the like.

According to step (e) of the process, the compound of formula (VII) is treated with a suitable acid such as, for instance, polyphosphoric or acetic acid, or even mixtures of acetic and hydrochloric acid; the Lewis acid is, for instance, zinc chloride, boron trifluoride, triethylaluminum or trifluoroacetic anhydride. Preferably, the reaction is carried out in the presence of polyphosphoric acid, by operating at temperatures ranging from about 0° C. to refluxing temperature and for a suitable time varying from about 30 minutes to about 96 hours.

The obtained compound of formula (I) may be then reacted, according to step (f) of the process, into another derivative of formula (I) by properly converting any desired R and R₁ group into another R and R₁ group, and/or into a pharmaceutically acceptable salt.

The operative conditions of step (f) are those previously reported in steps (b) and (c) of the former synthetic process.

The compounds of formula (V) and (VI) are known or can be easily prepared according to known methods.

From all of the above, it is clear to the skilled person that when preparing the compounds of formula (I) according to any process variant, which are all to be intended as within the scope of the present invention, optional functional groups within the starting materials, the reagents or the intermediates thereof and which could give rise to unwanted side reactions, need to be properly protected according to conventional techniques.

Likewise, the conversion of these latter into the free deprotected compounds may be carried out according to known procedures.

By analogy, pharmaceutically acceptable salts of the compounds of formula (I) or, alternatively, their free compounds from the salts thereof, may be all obtained according to conventional methods.

Likewise, it is also clear to the person skilled in the art that if a compound of formula (I), prepared according to the above processes, is obtained as an admixture of isomers, their separation into the single isomers of formula (I), carried out according to conventional techniques, is still within the scope of the present invention.

As formerly indicated, the compounds of formula (I) of the invention may be conveniently prepared according to combinatorial chemistry techniques widely known in the art, by accomplishing the aforementioned reactions between the several intermediates in a serial manner and by working under SPS conditions.

Accordingly, it is a further object of the present invention a library of two or more compounds of formula (I) 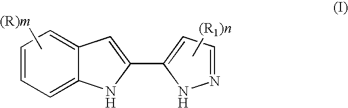
wherein
R is hydrogen, halogen, nitro, cyano, hydroxy, or it is a group optionally further substituted selected from straight or branched C₁-C₆ alkyl or C₁-C₆ alkoxy, C₃-C₆ cycloalkyl, aryl, heterocyclyl, or it is a group —NR′R″, —CONR′R″, —NR′COR″, —COOR′ or —SO₂NR′R″, wherein R′ and R″ are, the same or different and independently in each occasion, a hydrogen atom or a group optionally further substituted selected from straight or branched C₁-C₆ alkyl, C₃-C₆ cycloalkyl, aryl or heterocyclyl; or, taken together with the nitrogen atom to which they are attached, R′ and R″ may form a 5 or 6 membered nitrogen containing heterocycle, optionally comprising one additional heteroatom selected among N, O or S;
R₁ has the meanings above reported to R but other than hydroxy;
m is an integer from 1 to 4;
n is 1 or 2;
and the pharmaceutically acceptable salts thereof.

From all of the above, it is clear to the skilled person that once a library of pyrazolyl-indoles is thus prepared, for instance consisting of several hundreds of compounds of formula (I), the said library can be very advantageously used for screening towards given kinases, as formerly reported.

See, for a general reference to libraries of compounds and uses thereof as tools for screening biological activities, J. Med. Chem. 1999, 42, 2373-2382; and Bioorg. Med. Chem. Left. 10 (2000), 223-226.

### PHARMACOLOGY

The compounds of formula (I) are active as protein kinase inhibitors and are therefore useful, for instance, to restrict the unregulated proliferation of tumor cells.

In therapy, they may be used in the treatment of various tumors, such as those formerly reported, as well as in the treatment of other cell proliferative disorders such as psoriasis, vascular smooth cell proliferation associated with atherosclerosis and post-surgical stenosis and restenosis and in the treatment of Alzheimer's disease.

The inhibiting activity of putative cdk/cyclin inhibitors and the potency of selected compounds is determined through a method of assay based on the use of the SPA technology (Amersham Pharmacia Biotech).

The assay consists of the transfer of radioactivity labelled phosphate moiety by the kinase to a biotinylated substrate. The resulting 33P-labelled biotinylated product is allowed to bind to streptavidin-coated SPA beads (biotin capacity 130 pmol/mg), and light emitted was measured in a scintillation counter.

Inhibition Assay of cdk2/Cyclin A activity

Kinase reaction: 4 μM in house biotinylated histone H1 (Sigma # H-5505) substrate, 10 μM ATP (0.1 microCi P33γ-ATP), 1.1 nM Cyclin A/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 60 min at room temperature, the reaction was stopped by addition of 100 pi PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: inhibitors were tested at different concentrations ranging from 0.0015 to 10 μM. Experimental data were analyzed by the computer program GraphPad Prizm using the four parameter logistic equation:
y=bottom+(top-bottom)/(1+10\hat{ }((logIC50−x)*slope))
where x is the logarithm of the inhibitor concentration, y is the response; y starts at bottom and goes to top with a sigmoid shape.
Ki Calculation:

Experimental method: Reaction was carried out in buffer (10 mM Tris, pH 7.5, 10 mM MgCl₂, 0.2 mg/ml BSA, 7.5 mM DTT) containing 3.7 nM enzyme, histone and ATP (constant ratio of cold/labeled ATP 1/3000). Reaction was stopped with EDTA and the substrate captured on phosphomembrane (Multiscreen 96 well plates from Millipore). After extensive washing, the multiscreen plates were read on a top counter. Control (time zero) for each ATP and histone concentrations was measured.

Experimental design: Reaction velocities are measured at four ATP, substrate (histone) and inhibitor concentrations. An 80-point concentration matrix was designed around the respective ATP and substrate Km values, and the inhibitor IC50 values (0.3, 1, 3, 9 fold the Km or IC50 values). A preliminary time course experiment in the absence of inhibitor and at the different ATP and substrate concentrations allows the selection of a single endpoint time (10 min) in the linear range of the reaction for the Ki determination experiment.

Kinetic parameter estimates: Kinetic parameters were estimated by simultaneous nonlinear least-square regression using [Eq.1] (competitive inhibitor respect to ATP, random mechanism) using the complete data set (80 points):

$$v = \frac{Vm \cdot A \cdot B}{α \cdot Ka \cdot Kb + α \cdot Ka \cdot B + a \cdot Kb \cdot A + A \cdot B + α \cdot \frac{Ka}{Ki} \cdot I \cdot ( Kb + \frac{B}{β} )} \qquad [ Eq . 1 ]$$

where A=[ATP], B=[Substrate], I=[inhibitor], Vm=maximum velocity, Ka, Kb, Ki the dissociation constants of ATP, substrate and inhibitor respectively α and β the cooperativity factor between substrate and ATP binding and substrate and inhibitor binding respectively.

In addition the selected compounds are characterized on a panel of ser/thre kinases strictly related to cell cycle (cdk2/cyclin E, cdk1/cyclin B1, cdk5/p25, cdk4/cyclin D1), and also for specificity on MAPK, PKA, EGFR, IGF1-R, Aurora-2 and Cdc 7.

Inhibition Assay of cdk2/Cyclin E Activity

Kinase reaction: 10 μM in house biotinylated histone H1 (Sigma # H-5505) substrate, 30 μM ATP (0.3 microCi P³³γ-ATP), 4 ng GST-Cyclin E/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 60 min at room temperature, the reaction was stopped by addition of 100 pi PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of cdk1/Cyclin B1 Activity

Kinase reaction: 4 μM in house biotinylated histone H1 (Sigma # H-5505) substrate, 20 μM ATP (0.2 microCi P³³γ-ATP), 3 ng Cyclin B/CDK1 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of cdk5/p25 Activity

The inhibition assay of cdk5/p25 activity is performed according to the following protocol.

Kinase reaction: 10 μM biotinylated histone H1 (Sigma # H-5505) substrate, 30 μM ATP (0.3 microCi P³³γ-ATP), 15 ng CDK5/p25 complex, inhibitor in a final volume of 30 pI buffer (TRIS HCl 10 mM pH 7.5, MgCl₂ 10 mM, DTT 7.5 mM+0.2 mg/ml BSA) were added to each well of a 96 U bottom. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of cdk4/Cyclin D1 Activity

Kinase reaction: 0,4 uM μM mouse GST-Rb (769-921) (# sc-4112 from Santa Cruz) substrate, 10 μM ATP (0.5 μCi P³³γ-ATP), 100 ng of baculovirus expressed GST-cdk4/GST-Cyclin D1, suitable concentrations of inhibitor in a final volume of 50 pi buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 μl EDTA 120 mM.

Capture: 60 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled Rb fragment was detected by radioactivity counting in the Top-Count instrument.

IC50 determination: see above

Inhibition Assay of MAPK Activity

Kinase reaction: 10 μM in house biotinylated MBP (Sigma # M-1891) substrate, 15 μM ATP (0.15 microCi P33γ-ATP), 30 ng GST-MAPK (Upstate Biothecnology # 14-173), inhibitor in a final volume of 30 pI buffer (TRIS HCl 10 mM pH 7.5, MgCl₂ 10 mM, DTT 7.5 mM+0.2 mg/ml BSA) were added to each well of a 96 U bottom. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 pL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of PKA Activity

Kinase reaction: 10 μM in house biotinylated histone H1 (Sigma # H-5505) substrate, 10 μM ATP (0.2 microM P³³γ-ATP), 0.45 U PKA (Sigma # 2645), inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 90 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 pL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of EGFR Activity

Kinase reaction: 10 μM in house biotinyiated MBP (Sigma # M-1891) substrate, 2 μM ATP (0.04 microCi P³³γ-ATP), 36 ng insect cell expressed GST-EGFR, inhibitor in a final volume of 30 μl buffer (Hepes 50 mM pH 7.5, MgCl₂ 3 mM, MnCl₂ 3 mM, DTT 1 mM, NaVO₃ 3 μM, +0.2 mg/ml BSA) were added to each well of a 96 U bottom. After incubation for 20 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 pL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above

Inhibition Assay of IGF1—R Activity

The inhibition assay of IGF1-R activity is performed according to the following protocol.

Enzyme activation: IGF1-R must be activated by auto-phosphorylation before starting the experiment. Just prior to the assay, a concentrated enzyme solution (694 nM) is incubated for half a hour at 28° C. in the presence of 100 μM ATP and then brought to the working dilution in the indicated buffer.

Kinase reaction: 10 μM biotinylated IRS1 peptide (PRIMM) substrate, 0-20 μM inhibitor, 6 μM ATP, 1 microCi ³³P-ATP, and 6 nM GST-IGF1-R (pre-incubated for 30 min at room temperature with cold 60 μM cold ATP) in a final volume of 30 pI buffer (50 mM HEPES pH 7.9, 3 mM MnCl₂, 1 mM DTT, 3 μM NaVO₃) were added to each well of a 96 U bottom well plate. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

Inhibition assay of Aurora-2 activity

Kinase reaction: 8 μM biotinylated peptide (4 repeats of LRRWSLG), 10 μM ATP (0.5 uCi P³³γ-ATP), 7.5 ng Aurora 2, inhibitor in a final volume of 30 pI buffer (HEPES 50 mM pH 7.0, MgCl₂ 10 mM, 1 mM DTT, 0.2 mg/ml BSA, 3 μM orthovanadate) were added to each well of a 96 U bottom well plate. After 60 minutes at room temperature incubation, reaction was stopped and biotinylated peptide captured by adding 100 μl of bead suspension.

Stratification: 100 μl of CsCl2 5 M were added to each well and let stand 4 hour before radioactivity was counted in the Top-Count instrument.

IC50 determination: see above

Inhibition Assay of Cdc7/dbf4 Activity

The inhibition assay of Cdc7/dbf4 activity is performed according to the following protocol.

The Biotin-MCM2 substrate is trans-phosphorylated by the Cdc7/Dbf4 complex in the presence of ATP traced with γ³³-ATP. The phosphorylated Biotin-MCM2 substrate is then captured by Streptavidin-coated SPA beads and the extent of phosphorylation evaluated by β counting. The inhibition assay of Cdc7/dbf4 activity was performed in 96 wells plate according to the following protocol.

To each well of the plate were added:
10 μl substrate (biotinylated MCM2, 6 μM final concentration)
10 μl enzyme (Cdc7/Dbf4, 17.9 nM final concentration)
10 μl test compound (12 increasing concentrations in the nM to μM range to generate a dose-response curve)
10 μl of a mixture of cold ATP (2 μM final concentration) and radioactive ATP (1/5000 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 μM NaVO₃, 2 mM glycerophosphate and 0.2 mg/ml BSA. The solvent for test compounds also contained 10% DMSO.

After incubation for 60 minutes, the reaction was stopped by adding to each well 100 μl of PBS pH 7.4 containing 50 mM EDTA, 1 mM cold ATP, 0.1% Triton X100 and 10 mg/ml streptavidin coated SPA beads.

After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: see above.

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from about 10 to about 500 mg pro dose, from 1 to 5 times daily.

The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase II inhibitors and the like, optionally within liposomal formulations thereof.

If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

The present invention also includes pharmaceutical compositions comprising a compound of formula (I) or a pharmaceutically acceptable salt thereof in association with a pharmaceutically acceptable excipient (which can be a carrier or a diluent).

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatin, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulfates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerin and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

The following examples are herewith intended to better illustrate the present invention without posing any limitation to it.

### Experimental Part

General Methods

Flash chromatography was performed on silica gel (Merck grade 9385, 60 Å). HPLC/MS was performed on a Waters X Terra RP 18 (4.6×50 mm, 3.5 μm) column using a Waters 2790 HPLC system equipped with a 996 Waters PDA detector and a Micromass mod. ZQ single quadrupole mass spectrometer, equipped with an electrospray (ESI) ion source. Mobile phase A was ammonium acetate 5 mM buffer (pH 5.5 with acetic acid/acetonitrile 95:5), and Mobile phase B was H₂O/acetonitrile (5:95). Gradient from 10 to 90% B in 8 minutes, hold 90% B 2 min. UV detection at 220 nm and 254 nm. Flow rate 1 ml/min. Injection volume 10 μl. Full scan, mass range from 100 to 800 amu. Capillary voltage was 2.5 KV; Source temp. was 120° C.; Cone was 10 V. Retention Times (HPLC r.t.) are given in minutes at 220 nm or 254 nm. Mass are given as m/z ratio.

When necessary, compounds have been purified by Preparative HPLC on a Waters Symmetry C18 (19×50 mm, 5 um) column using a Waters preparative HPLC 600 equipped with a 996 Waters PDA detector and a Micromass mod. ZMD single quadrupole mass spectrometer, electrospray ionisation, positive mode. Mobile phase A was water 0.01% TFA, and Mobile phase B was acetonitrile. Gradient from 10 to 90% B in 8 min, hold 90% B 2 min. Flow rate 20 ml/m.

¹H-NMR spectroscopy was performed on a Mercury VX 400 operating at 400.45 MHz equipped with a 5 mm double resonance probe (1H {15N-31P} ID_PFG Varian).

### EXAMPLE 1

Ethyl 3-iodo-1H-pyrazole carboxylate

To a stirred mixture of 3.2 g (20 mmol) of ethyl 5-amino-1H-pyrazole-4-carboxylate in 95 mL of CH₂₁₂ at −10° C., 24 mL of isoamyl nitrite (180 mmol) were added during 30 minutes. The mixture was stirred two hours at 100° C. and, after cooling, it was concentrated under reduced pressure (first 10 mmHg then 0.1 mmHg). The residue was dissolved in ethyl acetate and the resulting solution washed with Na₂S₂O₅, HCl (1N) and water. Organic phase was separated, dried over Na₂SO₄, filtered and concentrated under reduced pressure to give a brown solid that was purified by column chromatography, eluting with 30% ethyl acetate in hexane to give 4.11 g (75%) of the title compound as a yellow solid.

¹H-NMR (DMSOd6), diagnostic signals (ppm): 13.71 (s, 1H), 8.25 (s, 1H), 4.19 (q, 2H), 1.26 (t, 3H)

[M+H]+=267

By operating as above described and by employing 5-amino-1H-pyrazole-4-carbonitrile in place of ethyl 5-amino-1H-pyrazole-4-carboxylate, the following compound was prepared:

3-iodo-1H-pyrazole-4-carbonitrile

¹H-NMR (DMSOd6), diagnostic signals (ppm): 13.71 (s, 1H), 8.51 (s, 1H),

[M+H]+=219

### EXAMPLE 2

Ethyl 3-iodo-1-{[2-(trimethylsilyl)ethoxy]methyl}-1H-pyrazole-4-carboxylate

Sodium Hydride (60% suspension in mineral oil, 0.3 g, 7.5 mmol) was suspended in dry THF (20 mL) under argon. The mixture was cooled at 0° C. and ethyl 5-iodo-1H-pyrazole-4-carboxylate (1.7 g, 7.5 mmol) in dry THF (20 mL) was added. The mixture was then stirred at room temperature for 1 hour and after cooling at 0° C. a solution of 2-(trimethylsilyl)ethoxymethyl chloride (1.3 mL, 7.5 mmol) in dry THF (5 mL) was added. After stirring at room temperature for 1.5 hours, water was added, the organic layer separated and the aqueous phase was extracted with ethyl acetate. The combined organic layers were dried over Na₂SO₄ and evaporated under vacuum affording 2.6 g of a crude oil which was chromatographed on silica gel eluting with 10% ethyl acetate in hexane. The fractions containing the title compound were evaporated giving rise to 0.4 g (13%) of the title compound as a colorless oil.

¹H-NMR (DMSOd6), diagnostic signals (ppm): 7.99 (s, 1H), 5.51 (s, 2H), 4.22 (q, 2H), 3.55 (t, 2H), 1.27 (t, 3H), 0.82 (t, 2H), −0.07 (s, 9H).

[M+H]+=397

By continuing the elution with 10% ethylacetate in hexane, 2.2 g (74%) of the following compound were obtained:

Ethyl 3-iodo-1-{[2-(trimethylsilyl)ethoxy]methyl}-1H-pyrazole-1-carboxylate

¹H-NMR (DMSOd6), diagnostic signals (ppm): 8.43 (s, 1H), 5.4 (s, 2H), 4.215 (q, 2H), 3.53 (t, 2H), 1.26 (t, 3H), 0.81 (t, 2H), −0.06 (s, 9H).

[M+H]+=397

### EXAMPLE 3

Ethyl 4-cyano-3-iodo-1H-pyrazole-1-carboxylate

A solution of 5-iodo-1H-pyrazole-4-carbonitrile (0.5 g 2.3 mmol) in diisopropylethylamine (0.88 g, 6.8 mmol) and anhydrous THF (20 mL) was cooled at 0° C. and ethyl chloroformate (0.3 g, 2.7 mmol) was added. The solution was stirred at 0° C. for 1 hour then partitioned between water and ethyl acetate The combined extracts were dried (Na₂SO₄) and evaporated in vacuo to give a crude solid. Chromatography eluting with 30% ethyl acetate in hexane gave 0.64 g (86%) of ethyl 4-cyano-3-iodo-1H-pyrazole-1-carboxylate as a white solid.

¹H-NMR (DMSOd6), diagnostic signals (ppm): 9.14 (s, 1H), 4.45 (q, 2H), 1.34 (t, 3H).

[M+H]+=292

### EXAMPLE 4

Tert-butyl 2-(4-(ethoxycarbonyl)-1 -{[2-(trimethylsilyl)ethoxy]methyl}-1H-pyrazol-3-yl)-1H-indole-1-carboxylate

To a stirred solution of ethyl 3-iodo-1{[2-(trimethylsilyl)ethoxy]methyl}-1H-pyrazole-4-carboxylate (50.0 mg, 0.13 mmol) and tetrakis-(triphenylphosphine)palladium (0) (15 mg, 0.013 mmol) in 1,2-dimethoxyethane (100 ml), 1-(tert-butoxycarbonyl)-1H-indol-2-ylboronic acid (52 mg, 0.2 mmol) and tallium carbonate (236 mg, 0.5 mmol) were added. The mixture was heated under argon at 80° C. for 12 hours, then cooled and partitioned between water and ethyl acetate. The combined extracts were dried (Na₂SO₄) and evaporated in vacuo to give a crude oil. Chromatography eluting with 20% ethyl acetate in hexane gave 5-[1-(tert-butoxycarbonyl)-1H-indol-2-yl]-1-{[2-(trimethylsilyl) ethoxy]methyl}-1H-pyrazole-4-carboxylic acid (21 mg, 35%) as a yellow oil.

¹H-NMR (DMSOd6), diagnostic signals (ppm):8.59 (s, 1H), 8.145 (d, 1H), 7.62 (d, 1H), 7.35 (t, 1H), 7.25 (t, 1H), 6.71 (s, 1H), 5.49 (s, 2H), 4.03 (q, 2H), 3.63 (q, 2H), 1.31 (s, 9H), 1.02 (t, 3H), 0.87 (q, 2H), −0.03 (s, 9H).

[M+H]+=487

### EXAMPLE 5

3-(1H-indol-2-yl)-1-{[2-(trimethylsilyl)ethoxy]methyl}-1H-pyrazole-4-carboxylic acid

A solution of tert-butyl 2-(4-(ethoxycarbonyl)-1-{[2-(trimethylsilyl)ethoxy]methyl}-1H-pyrazol-3-yl)-1H-indole-1-carboxylate (0.5 g, 1 mmol) in EtOH (10 mL) and NaOH 2N (2.5 mL) was refluxed for 2 hours. After cooling the solution was concentrated, treated with water, acidified with HCl and extracted with EtOAc. Organic phase was washed with water, dried (Na₂SO₄) and evaporated to give a crude solid. Purification by flash chromatography (CH₂Cl₂/CH₃OH 95:5) afforded 0.28 g of the title compound as a brown solid (70%).

¹H-NMR (DMSOd6), diagnostic signals (ppm): 12.75 (br, 1H), 11.65 (br, 1H), 8.55 (s, 1H), 7.56 (d, 1H), 7.45 (d, 1H), 7.28 (s, 1H), 7.10 (t, 1H), 7.98 (t, 1H), 5.49 (s, 2H), 3.62 (t, 2H), 0.87 (t, 2H), −0.04 (s, 9H).

[M+H]+=358

### EXAMPLE 6

3-(1H-indol-2-yl)-1H-pyrazole carboxylic acid

5-(1H-indol-2-yl)-1-{[2-(trimethylsilyl)ethoxy]methyl}-1H-pyrazole-4-carboxylic acid (100 mg 0.28 mmol) was treated with 4 M HCl in dioxane (3 mL) and MeOH (3 mL) at room temperature for 2 hours. The mixture was concentrated, basified with saturated sodium bicarbonate to pH 6 and filtered to give a crude solid. Purification by flash chromatography afforded 50.8 mg of the title compound as a brown solid (80%).

¹H-NMR (DMSOd6), diagnostic signals (ppm):11.55 (s, 1H), 8.15 (s, 1H), 7.57 (d, 1H), 7.47 (d, 1H), 7.2 (s, 1H), 7.12 (t, 1H), 7.02 (t, 1H).

[M+H]+=228

### EXAMPLE 7

Ethyl 3-(1H-indol-2-yl)-1H-pyrazole-4-carboxylate

A solution of 5-(1H-indol-2-yl)-1H-pyrazole-4-carboxylic acid (0.2 g, 0.9 mmol) in ethanol (5 mL) and H₂SO₄ 96% (0.3 mL) was refluxed overnight. After cooling the solution was concentrated, treated with water, basified with concentrated NaHCO₃ and extracted with EtOAc. Organic phase was washed with water, dried (Na₂SO₄) and evaporated to give a crude solid. Purification by flash chromatography (Hexane/EtOAc 1:1) afforded 0.19 g of the title compound as a white solid (85%)

¹H-NMR (DMSOd6), diagnostic signals (ppm): 13.77 (s, 1H), 11.5 (s, 1H), 8.32 (s, 1H), 7.60 (d, 1H), 7.52 (d, 1H), 7.29 (s, 1H), 7.15 (t, 1H), 7.04 (t, 1H), 4.33 (q, 2H), 1.34 (s, 1H).

[M+H]+=256

### EXAMPLE 8

3-(1H-indol-2-yl)-1H-pyrazole carbonitrile

By operating as reported for tert-butyl 2-(4-(ethoxycarbonyl)-1-{[2-(trimethylsilyl)ethoxy]methyl}-1H-pyrazol-3-yl)-1H-indole-1-carboxylate but employing ethyl 4-cyano-3-iodo-1H-pyrazole-1-carboxylate instead of 5-iodo-1-{[2-(trimethylsilyl) ethoxy]methyl}-1H-pyrazole-4-carboxylate, the title compound was obtained (50% yield).

¹H-NMR (DMSOd6), diagnostic signals (ppm): 13.89 (s, 1H), 11.59 (s, 1H), 8.68 (s, 1H), 7.59 (d, 1H), 7.41 (d, 1H), 7.12 (t, 1H), 7.03-6.98 (m, 2H).

[M+H]+=209

### EXAMPLE 9

3-(1H-indol-2-yl)-1H-pyrazole-4-carboxamide

To a mixture of 3-(1H-indol-2-yl)-1H-pyrazole-4-carboxylic acid (35 mg, 0.15 mmol), benzotriazol-1-yloxytris(pyrrolidino)phosphonium hexafluorophosphate (PYBOP, 120 mg, 0.23 mmol), 1-hydroxybenzotriazole (HOBt, 30 mg, 0.23 mmol) and N,N-diisopropylethylamine (105 mcL, 0.6 mmol) in DMF (2 mL), NH₄Cl (16.5 mg, 0.3 mmol) was added one pot. The resulting mixture was stirred at room temperature overnight The solvent was evaporated and the residue was dissolved in EtOAc. The EtOAc solution was extracted with 1 N HCl, washed with brine, extracted with saturated NaHCO₃, and dried over Na₂SO₄. Filtration and evaporation of the solvent gave a crude solid that was purified by preparative chromatography, affording the desired amide as a solid 30 mg (86%).

¹H-NMR (DMSOd6), diagnostic signals (ppm): 8.25 (br, 1H). 7.95 (br, 1H), 7.55(d, 1H), 7.47 (s, 1H), 7.46 (d, 1H), 7.13-7.03 (m, 2H), 7 (t, 1H).

[M+H]+=227

By operating in an analogous way and by using benzylamine in place of NH₄Cl, the following compound, as a white solid (80% yield), was obtained:

N-benzyl-5-(1H-indol-2-yl)-1H-pyrazole carboxamide

1H-NMR (DMSOd6), diagnostic signals (ppm): 13.9 (br, 1H), 9.06 (br, 1H), 8.33 (br, 1H), 7.55 (dd, 2H), 7.4-7.32 (m, 4H), 7.3-7.23(m, 1H), 7.2-7.07 (m, 2H), 7.03 (t, 1H), 4.57(d, 2H).

[M+H]+=317.

### EXAMPLE 10

4-{(2E)-2-[1-(1H-pyrazol-3-yl)ethylidene]hydrazino}benzonitrile

A mixture of 3-acetylpyrazole hydrochloride (0.6 g, 3.5 mmol) and 4-cyanophenylhydrazine hydrochloride (0.52 g, 3.5 mmol) in EtOH (4 ml) was heated to boiling for 5 hours. After cooling at 0° C. the precipitate was filtered and washed thoroughly with cold ethanol. After drying, the desired hydrazone was obtained as a yellow solid (0.7 g, 88% yield).

1H-NMR (DMSOd6), diagnostic signals (ppm): 9.82 (s, 1H), 7.67(d, 1H), 7.61 (s, 2H), 7.37 (d, 2H), 6.63 (d, 1H), 2.31 (s, 3H).

[M+H]+=226

By working in an analogous way the following compounds were obtained:

(1E)-1-(1H-pyrazol-3-yl)ethanone phenylhydrazone yellow solid (95% yield)

¹H-NMR (DMSOd6), diagnostic signals (ppm): 7.79 (s, 1H), 7.28 (d, 2H), 7.19 (t, 2H), 6.74 (t, 2H), 6.65 (s, 1H), 2.25 (s, 3H).

[M+H]+=201.

(1E)-1-(1H-pyrazol-3-yl)ethanone (4-chlorophenyl)hydrazone

yellow solid (80% yield)

¹H-NMR (DMSOd6), diagnostic signals (ppm): 9.31 (s, 1H), 7.64 (d, 1H), 7.29-7.21 (m, 4H), 6.57 (d, 1H), 2.25 (s, 3H).

[M+H]+=235.

(1E)-1-(1H-pyrazol-3-yl)ethanone (4-bromophenyl)hydrazone

yellow solid (90% yield)

¹H-NMR (DMSOd6), diagnostic signals (ppm): 9.32 (s, 1H), 7.64 (d, 1H), 7.35 (d, 2H), 7.22 (d, 2H), 6.58 (d, 1H), 2.25 (s, 3H).

[M+H]+=278.

Ethyl 3-{(2E)-2-[1-(1H-pyrazol-3-yl)ethylidene]hydrazino}benzoate

yellow solid (84% yield)

¹H-NMR (DMSOd6), diagnostic signals (ppm): 12.75(br, 1H), 8.4-6.57 (m, 6H), 4.32 (q, 3H), 2.3 (s, 3H), 2.75 (t, 3H).

[M+H]+=273.

### EXAMPLE 11

5-chloro-2-(1H-pyrazol-3-yl)-1H-indole

1-(1H-pyrazol-3-yl)ethanone (4-chlorophenyl) (0.5 g, 20.4 mmol) was added to polyphosphoric acid (5 mL) and the thick mixture was stirred at 90° C. for 2 hours. Heating was removed and the mixture was let to cool to room temperature before pouring it into 25 mL of stirred water. After 30 minutes stirring, the precipitate was filtered and dried under reduced pressure to give 0.2 g (43%) of the title compound as a yellow solid

¹H-NMR (DMSOd6), diagnostic signals (ppm): 13.02 (s, 1H), 11.60 (s, 1H), 7.83(s, 1H), 7.55(s, 1H), 7.39(d, 1H), 7.07(sd, 1H), 6.73(d, 2H).

[M+H]+=218

By operating in an analogous way and by using (1E)-1-(1H-pyrazol-3-yl)ethanone phenylhydrazone in place of 1-(1H-pyrazol-3-yl)ethanone (4-chlorophenyl), the following compound was obtained:

2-(1H-pyrazol-3-yl)-1H-indole

white solid in 80% yield

¹H-NMR (DMSOd6), diagnostic signals (ppm): 12.9 (s, 1H), 11.32 (s, 1H), 7.78 (s, 1H), 7.47 (d, 1H), 7.36 (d, 1H), 7.02 (t, 1H), 6.94(t, 1H), 6.67 (s, 1H), 6.68 (s, 1H).

[M+H]+=184.

By operating in an analogous way and by using 1-(1H-pyrazol-3-yl)ethanone (4-bromophenyl) in place of 1-(1H-pyrazol-3-yl)ethanone (4-chlorophenyl), the following compound was obtained:

5-bromo-2-(1H-pyrazol-3-yl)-1H-indole

yellow solid in 75% yield

¹H-NMR (DMSOd6), diagnostic signals (ppm): 13.03 (s, 1H), 11.61 (s, 1H), 7.82(s, 1H), 7.70 (s, 1H), 7.35(d, 1H), 7.18(s, 1H), 6.74(s, 1H), 6.73 (s, 2H).

[M+H]+=263.

### EXAMPLE 12

2-(1H-pyrazol-3-yl)-1H-indole-5-carboxamide

By starting from 4-{(2E)-2-[1-(1H-pyrazol-3-yl)ethylidene]hydrazino}benzonitrile as prepared in example 10, and by working as described in example 11 in the presence of polyphosphoric acid, the title compound was obtained as a yellow solid (75% yield). 1H-NMR (DMSOd6), diagnostic signals (ppm): 13.01 (s, 1H), 12.4 (s, 1H), 11.76 (s, 1H), 8.21 (s, 1H), 7.81 (s, 1H), 7.71 (dd, 1H), 7.44 (d, 1H), 6.89 (s, 1H), 6.75(d, 1H).

[M+H]+=227.

### EXAMPLE 13

25 Ethyl 2-(1H-pyrazol-3-yl)-1H-indole carboxylate

A stirred mixture of ethyl 3-{(2E)-2-[1-(1H-pyrazol-3-yl)ethylidene]hydrazino}benzoate (18 g, 66 mmol) in poliphosphoric acid (200 g) was slowly heated at 80-85° C. After keeping for 15 minutes at this temperature, the resulting yellow cream was rapidly treated with iced water. The solid was filtered off, washed with water and dissolved in ethylacetate and washed with 0.1 M NaOH, then with brine. After drying over Na₂SO₄, the solvent was removed and the residue carefully chromatographed on silica gel eluting with CH₂Cl₂/Et₂O 8/2. The fractions containing the compound were pooled, the solvent removed and the residue crystallized twice from Et₂O to give 8.3 g of the title compound (51% yield).

¹H-NMR (DMSOd6), diagnostic signals (ppm): 13.10 (br, 1H), 11.80 (br, 1H), 8.07-6.78 (m, 6H), 4.34 (q, 2H), 1.38 (t, 3H).

[M+H]+=256

By continuing the elution with the mixture CH₂Cl₂/Et₂O 8/3, pooling the fraction and crystallizing trice from Et₂O, 3.7 g of the following compound were obtained (21% yield):

Ethyl 2-(1H-pyrazol-3-yl)-1H-indole-6-carboxylate

¹H-NMR (DMSOd6), diagnostic signals (ppm): 13.20 (br, 1H), 11.60 (br, 1H), 8.17-6.43 (m, 6H), 4.53 (q, 2H), 1.41 (t, 3H).

[M+H]+=256

### EXAMPLE 14

2-(1H-pyrazol-3-yl)-1H-indole 4 carboxylic acid

A stirred solution of ethyl 2-(1H-pyrazol-3-yl)-1H-indole-4-carboxylate (4 g, 15.7 mmol), 2 M NaOH (16 mL) in EtOH (100 mL) was refluxed for 4 hours. The solvent was partially removed and then, after dilution with ethylacetate, the reaction mixture was treated with 1 M HCl (33 mL). After extraction, the organic phase was thoroughly washed with brine and dried over Na₂SO₄. After removal of the solvent, the solid was crystallized from methanol to furnish 2.8 g (77% yield) of the title compound.

¹H-NMR (DMSOd6), diagnostic signals (ppm): 13.1 (br, 1H), 12.6 (br, 2H), 8.05-6.77 (m, 6H).

[M+H]+=228.

By working in an analogous way and by using ethyl 2-(1H-pyrazol-3-yl)-1H-indole-6-arboxylate in place of ethyl 2-(1H-pyrazol-3-yl)-1H-indole-4-carboxylate, the following compound was obtained (69% yield):

2-(1H-pyrazol-3-yl)-1H-indole-6-carboxylic acid

¹H-NMR (DMSOd6), diagnostic signals (ppm): 13.23 (br, 1H), 12.7 (br, 2H), 8.13-6.85 (m, 6H).

[M+H]+=228.

### EXAMPLE 15

2-(1H-pyrazol-3-yl)-1H-indole-5-carboxylic acid

A solution of 5-(1H-indol-2-yl)-1H-pyrazole-4-carboxamide (6 g, 26.5 mmol) in 220 mL of NaOH 20% (1.34 mol) and 200 mL of methanol was refluxed for 7 hours. The solvent was partially removed then treated with HCl 25% (175 mL). The obtained solid was filtered, thoroughly washed with water and then dried to give 5 g (83%) of the title compound as a yellowish solid.

¹H-NMR (DMSOd6), diagnostic signals (ppm): 13.01 (s, 1H), 11.62 (s, 1H), 8.12 (s, 1H), 7.83(s, 2H), 7.65 (d, 1H), 7.39 (d, 1H), 7.05 (s, 1H), 6.82 (s, 1H), 6.75 (s, 1H).

[M+H]+=228

### EXAMPLE 16

Methyl 2-(1H-pyrazol-3-yl)-1H-indole-5-carboxylate

A solution of 2-(1H-pyrazol-3-yl)-1H-indole-5-carboxylic acid (4.5 g, 20 mmol) in MeOH (50 mL) and H₂SO₄ 96% (1.5 mL) was refluxed overnight. After cooling the solution was concentrated, treated with water, basified with NaOH 2N and extracted with EtOAc. Organic phase was washed with water, dried (Na₂SO₄) and evaporated to give a crude solid. Purification by flash chromatography (hexane/EtOAc 4:6) afforded 4 g of the title compound as a yellow solid (84%).

1H-NMR (DMSOd6), diagnostic signals (ppm): 13.05 (s, 1H), 11.81 (s, 1H), 8.23 (s, 1H), 7.85 (s, 1H), 7.72 (d, 1H), 7.46 (d, 1H), 6.89 (s, 1H), 6.76 (d, 1H), 3.86 (s, 3H).

[M+H]+=242.

### EXAMPLE 17

20 By working as described in example 9 and by starting from the suitable carboxylic acid derivative, e.g. 2-(1H-pyrazol-3-yl)-1H-indole-4-carboxylic acid, -5-carboxylic acid or -6-carboxylic acid, the following carboxamide derivatives were obtained:

2-(1H-pyrazol-3-yl)-1H-indole-4-carboxamide

37% yield.

¹H-NMR (DMSOd6), diagnostic signals (ppm): 12.98 (br, 1H), 11.57 (br, 1H), 7.78 (m, 3H), 7.51-7.45 (m, 2H), 7.11 (m, 2H), 6.65 (d, 1H).

[M+H]+=227.

2-(1H-pyrazol-3-yl)-1H-indole-6-carboxamide

44% yield

¹H-NMR (DMSOd6), diagnostic signals (ppm): 12.94 (br, 1H), 11.55 (br, 1H), 7.79 (m, 3H), 7.53-7.47 (m, 2H), 7.17 (m, 2H), 6.69 (d, 1H).

[M+H]+=227.

N-ethyl-2-(1H-pyrazol-3-yl)-1H-indole-5-carboxamide

¹H-NMR (DMSOd6), diagnostic signals (ppm): 13.09 (s, 1H), 11.62 (s, 1H), 8.28 (t, 1H), 8.08 (s, 1H), 7.79 (s, 1H), 7.62 (d, 1H), 7.40 (d, 1H), 6.84 (d, 1H), 6.75 (d. 1H), 3.32 (m, 2H) 1.14 (q, 3H).

[M+H]+=256.

N-isobutyl-2-(1H-pyrazol-3-yl)-1H-indole-4-carboxamide

75% yield

¹H-NMR (DMSOd6), diagnostic signals (ppm): 13.01 (br, 1H), 11.56 (br, 1H), 8.20 (t, ₁H), 7.83-6.64 (m, 6H), 3.15 (d, 2H), 1.85 (m, 1H), 0.96 (d, 6H).

[M+H]+=283.

N-isobutyl-2-(1H-pyrazol-3-yl)-1H-indole-5-carboxamide

¹H-NMR (DMSOd6), diagnostic signals (ppm): 13.02 (s, 1H), 11.62 (s, 1H), 8.28 (t, 1H), 8.09 (s, 1H), 7.84 (s, 1H), 7.62(dd, 1H), 7.40(d, 1H), 6.84 (s, 1H) 6.75 (s, 1H), 3.11 (t, 2H), 1.88 (m, 1H), 0.93 (s, 3H), 0.91 (s, 3H).

[M+H]+=283.

N-isobutyl-2-(1H-pyrazol-3-yl)-1H-indole-6-carboxamide 67% yield.

¹H-NMR (DMSOd6), diagnostic signals (ppm): 13.01 (br, 1H), 11.56 (br, 1H), 8.20 (t, 1H), 7.83-6.64 (m, 6H), 3.15 (d, 2H), 1.89 (m, 1H), 0.93 (d, 6H).

[M+H]+=283.

N-(3-hydroxypropyl)-2-(1H-pyrazol-3-yl)-1H-indole arboxamide

53% yield

¹H-NMR (DMSOd6), diagnostic signals (ppm): 13.03 (br, 1H), 11.57 (br, 1H), 8.17 (t, 1H), 7.83-6.74 (m, 6H), 4.52 (t, 1H), 3.53 (m, 2H), 3.33 (m, 2H), 1.74 (m, 2H), 3.15 (d, 2H).

[M+H]+=285

N-(3-hydroxypropyl)-2-(1H-pyrazol-3-yl)-1H-indole-5-carboxamide

¹H-NMR (DMSOd6), diagnostic signals (ppm): 13.00 (br, 1H), 11.61 (br, 1H), 8.27 (m, 1H), 8.07 (br, 1H), 7.84 (m, 1H), 7.59 (m, 1H), 7.41 (m, 1H), 6.82 (br, 1H), 4.49 (br, 1H), 3.33 (m, 4H), 1.71 (m, 2H)

[M+H]+=285.

N-(3-hydroxypropyl)-2-(1H-pyrazol-3-yl)-1H-indole-6-carboxamide

71% yield

¹H-NMR (DMSOd6), diagnostic signals (ppm): 13.13 (br, 1H), 11.59 (br, 1H), 8.13 (t, 1H), 7.91-6.75 (m, 6H), 4.56 (t, 1H), 3.59 (m, 2H), 3.23 (m, 2H), 1.76 (m, 2H), 3.18(d, 2H).

[M+H]+=285.

N-(2-methoxyethyl)-2-(1H-pyrazol-3-yl)-1H-indole-5-carboxamide

¹H-NMR (DMSOd6), diagnostic signals (ppm):11.64 (s, 1H), 8.32 (t, 1H), 8.10 (s, 1H), 7.79 (d, 1H), 7.63 (dd, 1H), 7.40 (d, 1H), 6.845 (d, 1H), 6.755 (d, 1H), 3.51-3.30 (m, 7H).

[M+H]+=285.

N-(2-methoxyethyl)-2-(1H-pyrazol-3-yl)-1H-indole-6-carboxamide

75% yield.

¹H-NMR (DMSOd6), diagnostic signals (ppm): 13.03 (br, 1H), 11.68 (br, 1H), 8.36 (t, 1H), 7.93-6.77 (m, 6H), 3.49 (t, 2H), 3.30 (t, 2H), 3.38 (s, 3H).

[M+H]+=285.

N-cyclopentyl-2-(1H-pyrazol-3-yl)-1H-indole-5-carboxamide

¹H-NMR (DMSOd6), diagnostic signals (ppm): 11.61 (br, 2H), 8.11 (br, 1H), 8.09 (br, 1H), 7.79 (m, 1H), 7.63 (m, 1H), 7.40 (m, 1H), 6.84 (br, 1H), 6.75 (m, 1H), 4.26 (m, 1H), 1.90, 1.75, 1.56 (m, 8H)

[M+H]+=295.

N-(4-hydroxybutyl)-2-(1H-pyrazol-3-yl)-1H-indole-5-carboxamide

¹H-NMR (DMSOd6), diagnostic signals (ppm): 13.0 (s, 1H), 11.61 (s, 1H), 8.27 (t, 1H), 8.07 (s, 1H), 7.83 (s, 1H), 7.61 (d, 1H), 7.40 (d, 1H), 6.82 (s, 1H), 6.75 (d, 1H), 4.41 (s, 1H), 3.45-3.28 (m, 4H), 1.62-1.55 (m, 2H), 1.52-1.47 (m, 2H).

[M+H]+=299.

N-(4-hydroxybutyl)-2-(1H-pyrazol-3-yl)-1H-indole-6-carboxamide

80% yield

¹H-NMR (DMSOd6), diagnostic signals (ppm): 13.03 (br, 1H), 11.66 (br, 1H), 8.32 (t, 1H), 7.92-6.76 (m, 6H), 3.45 (t, 2H), 3.30 (t, 2H), 1.51 (m, 4H).

[M+H]+=299

N-(2-furylmethyl)-2-(1H-pyrazol-3-yl)-1H-indole-4-carboxamide

83% yield

¹H-NMR (DMSOd6), diagnostic signals (ppm): 13.00 (br, 1H), 11.60 (br, 1H), 8.17 (t, 1H), 7.84-6.31 (m, 9H), 4.77 (d, 2H), 3.33 (m, 5H), 1.26 (m, 4H).

[M+H]+=307.

N-(2-furylmethyl)-2-(1H-pyrazol-3-yl)-1H-indole-5-carboxamide

¹H-NMR (DMSOd6), diagnostic signals (ppm): 13.25 (s, 1H), 11.65 (s, 1H), 8.78 (t, 1H), 8.13 (d, 1H), 7.79 (d, 1H), 7.65 (dd, 1H), 7.41 (d, 1H), 6.85 (d, 1H), 6.755 (d, 1H), 6.41 (q, 1H), 6.28 (dd, 1H), 4.49 (d, 2H).

[M+H]+=307.

N-(2-furylmethyl)-2-(1H-pyrazol-3-yl)-1H-indole-6-carboxamide

76% yield

¹H-NMR (DMSOd6), diagnostic signals (ppm): 13.05 (br, 1H), 11.58 (br, 1H), 8.19 (t, 1H), 7.82-6.37 (m, 9H), 4.73 (d, 2H), 3.31 (m, 5H), 1.28 (m, 4H).

[M+H]+=307.

4-(piperidin-1-ylcarbonyl)-2-(1H-pyrazol-3-yl)-1H-indole

66% yield

¹H-NMR (DMSOd6), diagnostic signals (ppm): 13.03 (br, 1H), 11.59 (br, 1H), 7.43-6.65 (m, 6H), 3.33 (m, 4H), 1.33 (m, 6H).

[M+H]+=295.

5-(piperidin-1-ylcarbonyl)-2-(1H-pyrazol-3-yl)-1H-indole

¹H-NMR (DMSOd6), diagnostic signals (ppm): 13.00 (br, 1H), 11.58 (br, 1H), 7.85 (m, 1H), 7.55 (br, 1H), 7.40 (br, 1H), 7.11 (m, 1H), 6.79 (br, 1H), 6.74 (br, 1H), 3.50 (br, 4H), 1.65 (m, 2H), 1.53 (br, 4H)

[M+H]+=295.

6-(piperidin-1-ylcarbonyl)-2-(1H-pyrazol-3-yl)-1H-indole

82% yield

¹H-NMR (DMSOd6), diagnostic signals (ppm): 13.12 (br, 1H), 11.56 (br, 1H), 7.39-6.62 (m, 6H), 3.31 (m, 4H), 1.35 (m, 6H).

[M+H]+=295.

4-[(4-methylpiperazin-1-yl)carbonyl]-2-(1H-pyrazol-3-yl)-1H-indole

71% yield

¹H-NMR (DMSOd6), diagnostic signals (ppm): 13.01 (br, 1H), 11.61 (br, 1H), 7.83-6.68 (m, 6H), 3.34 (m, 4H), 2.52 (m, 4H), 2.22 (s, 3H).

[M+H]+=310.

5-[(4-methylpiperazin-1-yl)carbonyl]-2-(1H-pyrazol-3-yl)-1H-indole

¹H-NMR (DMSOd6), diagnostic signals (ppm):13.00 (s, 1H), 11.62 (s, 1H), 7.82 (s, 1H), 7.58 (s, 1H), 7.42 (d, 1H), 7.12 (d, 1H), 6.81 (s, 1H), 6.745 (d, 1H), 3.60-3.50 (m, 4H), 2.43-2.34(m, 7H).

[M+H]+=310.

6-[(4-methylpiperazin-1-yl)carbonyl]-2-(1H-pyrazol-3-yl)-1H-indole

63% yield

¹H-NMR (DMSOd6), diagnostic signals (ppm): 13.06(br, 1H), 11.59 (br, 1H), 7.81-6.73 (m, 6H), 3.34 (m, 4H), 2.55 (m, 4H), 2.21 (s, 3H).

[M+H]+=310.

2-(1H-pyrazol-3-yl)-N-(tetrahydrofuran-2-ylmethyl)-1H-indole-5-carboxamide

¹H-NMR (DMSOd6), diagnostic signals (ppm): 13.01 (br, 1H), 11.62 (br, 1H), 8.32 (m, 2H), 8.10 (br, 1H), 7.83 (br, 1H), 7.62 (m, 1H), 7.41 (m, 1H), 7.36 (m, 5H), 6.83 (br, 1H), 6.75 (m, 1H), 4.02 (m, 1H), 3.80 (m, 1H), 3.66 (m, 1H), 1.85 (m, 2H), 1.63 (m, 2H)

[M+H]+=311

2-(1H-pyrazol-3-yl)-N-(tetrahydrofuran-2-ylmethyl)-1H-indole-6-carboxamide

68% yield

¹H-NMR (DMSOd6), diagnostic signals (ppm): 13.04 (br, 1H), 11.67 (br, 1H), 8.36 (m, 1H), 7.93-6.77 (m, 6H), 4.02 (m, 1H), 3.66 (m, 2H), 3.40 (m, 4H).

[M+H]+=311.

1-{[2-(1H-pyrazol-3-yl)-1H-indol yl]carbonyl}piperidin-4-ol

65% yield

¹H-NMR (DMSOd6), diagnostic signals (ppm): 13.00 (br, 1H), 11.60 (br, 1H), 8.17 (t, 1H), 7.82-6.64 (m, 6H), 4.77 (d, 1H), 3.33 (m, 5H), 1.26 (m, 4H).

[M+H]+=311.

1-{[2-(1H-pyrazol-3-yl)-1H-indol-5-yl]carbonyl}piperidin-4-ol

¹H-NMR (DMSOd6), diagnostic signals (ppm): 13.00 (br, 1H), 11.58 (br, 1H), 7.83 (br, 1H), 7.56 (br, 1H), 7.41 (m, 1H), 7.11 (m, 1H), 6.78 (br, 1H), 6.74 (m, 1H), 4.78 (br, 1H), 3.74 (m, 1H), 3.34 (m, 4H), 1.76 (m, 2H), 1.39 (m, 2H).

[M+H]+=311

1-{[2-(1H-pyrazol-3-yl)-1H-indol-6-yl]carbonyl}piperidin-4-ol 53% yield

¹H-NMR (DMSOd6), diagnostic signals (ppm): 13.03 (br, 1H), 11.58 (br, 1H), 8.15 (t, 1H), 7.87-6.61 (m, 6H), 4.75 (d, 1H), 3.35 (m, 5H), 1.28 (m, 4H).

[M+H]+=311.

N-[3-(dimethylamino)propyl]-2-(1H-pyrazol-3-yl)-1H-indole-4-carboxamide

1H-NMR (DMSOd6), diagnostic signals (ppm): 13.02 (s, 1H), 11.58 (s, 1H), 8.28 (s, 1H), 7.84 (s, 1H), 7.51 (d, 1H), 7.37 (d, 1H), 7.28-7.09 (m, 2H), 6.75 (d, 1H), 3.33 (m, 2H), 2.38 (m, 2H), 2.22 (s, 6H), 1.73 (m, 2H.

[M+H]+=312.

N-[3-(dimethylamino)propyl]-2-(1H-pyrazol-3-yl)-1H-indole-5-carboxamide

¹H-NMR (DMSOd6), diagnostic signals (ppm): 13.01 (br, 1H), 11.62 (br, 1H), 8.35 (m, 1H), 8.07 (br, 1H), 7.83 (br, 1H), 7.59 (m, 1H), 7.41 (m, 1H), 6.82 (br, 1H), 6.75 (m, 1H), 3.34 (m, 2H), 2.35 (m, 2H), 2.21 (s, 6H), 1.70 (m, 2H).

[M+H]+=312.

N-[3-(dimethylamino)propyl]-2-(1H-pyrazol-3-yl)-1H-indole-6-carboxamide

¹H-NMR (DMSOd6), diagnostic signals (ppm): 13.04 (br, 1H), 11.67 (br, 1H), 8.41 (t, 1H), 7-91-6.77 (m, 6H), 3.34 (m, 2H), 2.33 (m, 2H), 2.20 (s, 6H), 1.70 (m, 2H).

[M+H]+=312.

N-benzyl-2-(1H-pyrazol-3-yl)-1H-indole-4-carboxamide

71% yield

¹H-NMR (DMSOd6), diagnostic signals (ppm): 13.02(br, 1H), 11.60 (br, 1H), 8.80 (t, 1H), 7.83-6.32 (m, 11H), 4.54 (d, 2H).

[M+H]+=317.

N-benzyl-2-(1H-pyrazol-3-yl)-1H-indole-5-carboxamide

¹H-NMR (DMSOd6), diagnostic signals (ppm): 13.01 (br, 1H), 11.64 (br, 1H), 8.88 (m, 1H), 8.15 (br, 1H), 7.83 (br, 1H), 7.66 (m, 1H), 7.43 (m, 1H), 7.36 (m, 5H), 6.84 (br, 1H), 6.75 (m, 1H), 4.52 (m, 2H)

[M+H]+=317.

N-benzyl-2-(1H-pyrazol-3-yl)-1H-indole-6-carboxamide

73% yield

¹H-NMR (DMSOd6), diagnostic signals (ppm): 13.05(br, 1H), 11.62 (br, 1H), 8.70 (t, 1H), 7.85-6.41 (m, 11H), 4.58 (d, 2H). [M+H]+=317. 2-(1H-pyrazol-3-yl)-N-(pyridin-4-ylmethyl)-1H-indole-5-carboxamide

¹H-NMR (DMSOd6), diagnostic signals (ppm):13.02 (s, 1H), 11.67 (s, 1H), 8.97 (t, 1H), 8.52 (dd, 2H), 8.17 (s, 1H), 7.84 (s, 1H), 7.68 (d, 1H), 7.44 (d, 1H), 7.34 (dd, 2H), 6.85 (s, 1H), 6.76 (s, 1H), 4.53 (d, 2H). [M+H]+=318. 2-(1H-pyrazol-3-yl)-N-(pyridin ylmethyl)-1H-indole-6-carboxamide 60% yield

¹H-NMR (DMSOd6), diagnostic signals (ppm): 13.05 (br, 1H), 11.76 (br, 1H), 9.01 (t, 1H), 8.52-6.78 (m, 10H), 4.53 (d, 2H). 2-(1H-pyrazol-3-yl)-N-(2-pyridin-2-ylethyl)-1H-indole-5-carboxamide

¹H-NMR (DMSOd6), diagnostic signals (ppm): 13.00 (br, 1H), 11.62 (br, 1H), 8.53 (m, 1H), 8.40 (br, 1H), 8.06 (br, 1H), 7.83 (br, 1H), 7.72 (m, 1H), 7.59 (m, 1H), 7.41 (m, 1H), 7.32 (m, 1H), 7.24 (m, 1H), 6.82 (br, 1H), 6.75 (br, 1H), 3.65 (m, 2H), 3.04 (m, 2H). [M+H]+=332. 2-(1H-pyrazol-3-yl)-N-(2-pyridin-2-ylethyl)-1H-indole-6-carboxamide 77% yield

¹H-NMR (DMSOd6), diagnostic signals (ppm): 13.04 (br, 1H), 11.68 (br, 1H), 8.54 (d, 1H), 8.44 (m, 1H), 7.91-6.77 (m, 9H), 3.66 (m, 2H), 3.04 (m, 2H). [M+H]+=332.

N-(4-methoxyphenyl)-2-(1H-pyrazol-3-yl)-1H-indole-4-carboxamide 58% yield

¹H-NMR (DMSOd6), diagnostic signals (ppm): 13.21(br, 1H), 11.64 (br, 1H), 10.03 (s, 1H), 7.84-6.67 (m, 10H), 3.77 (s, 3H). [M+H]+=333.

N-(4-methoxyphenyl)-2-(1H-pyrazol-3-yl)-1H-indole-5-carboxamide

¹H-NMR (DMSOd6), diagnostic signals (ppm): 13.03 (br, 1H), 11.70 (br, 1H), 9.99 (s, 1H), 8.21 (br, 1H), 7.85 (br, 1H), 7.71 (m, 3H), 7.48 (m, 1H), 6.95 (m, 2H), 6.88 (br, 1H), 6.77 (m, 1H), 3.77 (s, 3H). [M+H]+=333.

N-(4-methoxyphenyl)-2-(1H-pyrazol-3-yl)-1H-indole-6-carboxamide 66% yield

¹H-NMR (DMSOd6), diagnostic signals (ppm): 13.06(s, 1H), 11.74 (s, 1H), 10.03 (s, 1H), 8.02 (s, 1H) 7.86 (s, 1H) 7.73-7.59 (m, 4H) 6.93-6.92 (m, 2H) 6.82 (s, 1H) 6.78 (d, 1H), 3.79 (s, 3H). [M+H]+=333.

N-(4-fluorobenzyl)-2-(1H-pyrazol-3-yl)-1H-indole-5-carboxamide

¹H-NMR (DMSOd6), diagnostic signals (ppm): 13.01 (br, 1H), 11.65 (br, 1H), 8.89 (m, 1H), 8.14 (br, 1H), 7.84 (br, 1H), 7.65 (m, 1H), 7.41 (m, 1H), 7.39 (m, 2H), 7.16 (m, 2H), 6.83 (br, 1H), 6.75 (m, 1H), 4.48 (m, 2H) [M+H]+=335.

N-(4-fluorobenzyl)-2-(1H-pyrazol-3-yl)-1H-indole-6-carboxamide 85% yield

¹H-NMR (DMSOd6), diagnostic signals (ppm): 13.04 (br, 1H), 11.70 (br, 1H), 8.93 (t, 1H), 7.97-6.77 (m, 10H), 4.50 (d, 2H). [M+H]+=335.

N-[3-(1H-imidazol-1-yl)propyl]-2-(1H-pyrazol-3-yl)-1H-indole-5-carboxamide

¹H-NMR (DMSOd6), diagnostic signals (ppm):13.01 (s, 1H), 11.64 (s, 1H), 8.36 (t, 1H), 8.09 (s, 1H), 7.84 (s, 2H), 7.62 (d, 1H), 7.41 (d, 1H), 7.30 (s, 1H), 7.30 (s, 1H), 6.84 (s, 1H), 6.755 (d, 1H), 4.08 (t, 2H), 3.30-3.26 (m, 2H), 2.04-1.97 (m, 2H).

[M+H]+=335.

N-(2-anilinoethyl)-2-(1H-pyrazol-3-yl)-1H-indole-4-carboxamide 47% yield

¹H-NMR (DMSOd6), diagnostic signals (ppm): 13.02(br, 1H), 11.59 (br, 1H), 8.29 (m, 1H), 7.84-6.55 (m, 11H), 5.73 (m, 1H), 3.35-3.32 (m, 4H).

[M+H]+=346.

N-(2-anilinoethyl)-2-(1H-pyrazol-3-yl)-1H-indole-5-carboxamide

¹H-NMR (DMSOd6), diagnostic signals (ppm): 13.01 (s, 1H), 11.63 (s, 1H), 8.41 (t, 1H), 8.10 (s, 1H), 7.84 (s, 1H), 7.63 (d, 1H), 7.41 (d, 1H), 7.10 (t, 2H), 6.83 (s, 1H), 6.76 (d, 1H), 6.64 (d, 2H), 6.54 (t, 1H), 5.71 (t, 1H), 3.47 (q, 2H), 3.22 (q, 2H).

[M+H]+=346.

N-(2-anilinoethyl)-2-(1H-pyrazol-3-yl)-1H-indole-6-carboxamide

59% yield

¹H-NMR (DMSOd6), diagnostic signals (ppm): 13.05(br, 1H), 11.61 (br, 1H), 8.32 (m, 1H), 7.86-6.63 (m, 11H), 5.75 (m, 1H), 3.37-3.31 (m, 4H).

[M+H]+=346.

N-(4-methoxy-2-methylphenyl)-2-(1H-pyrazol-3-yl)-1H-indole-5-carboxamide

¹H-NMR (DMSOd6), diagnostic signals (ppm): 12.99 (s, 1H), 11.69 (s, 1H), 9.60 (s, 1H), 8.24 (s, 1H), 7.81 (s, 1H), 7.74(dd, 1H), 7.46 (d, 1H), 7.23 (d, 1H), 6.89 (s, 1H), 6.86 (d, 1H), 6.79 (dd, 1H), 6.775 (d, 1H), 3.77 (s, 3H), 2.24 (s, 3H).

[M+H]+=347.

N-(2,5-difluorobenzyl)-2-(1H-pyrazol-3-yl)-1H-indole-5-carboxamide

¹H-NMR (DMSOd6), diagnostic signals (ppm): 13.02 (br, 1H), 11.67 (br, 1H), 8.90 (m, 1H), 8.16 (br, 1H), 7.83 (br, 1H), 7.66 (m, 1H), 7.45 (m, 1H), 7.26 (m, 1H), 7.15 (m, 2H), 6.86 (br, 1H), 6.76 (m, 1H), 4.52 (m, 2H)

[M+H]+=353.

N-(3-morpholin-4-ylpropyl)-2-(1H-pyrazol-3-yl)-1H-indole-5-carboxamide

¹H-NMR (DMSOd6), diagnostic signals (ppm): 11.68 (br, 2H) 8.50 (m, 1H), 8.10 (br, 1H), 7.80 (br, 1H), 7.62 (m, 1H), 7.51 (br, 1H), 6.85 (br, 1H), 6.75 (m, 1H), 3.35 (br, 12H), 1.95 (m, 2H).

[M+H]+=354.

5-[(4-benzylpiperazin-1-yl)carbonyl]-2-(1H-pyrazol-3-yl)-1H-indole

¹H-NMR (DMSOd6), diagnostic signals (ppm): 13.00 (br, 1H), 11.60 (br, 1H), 7.83 (br, 1H), 7.57 (br, 1H), 7.41 (m, 1H), 7.34 (m, 5H), 7.13 (m, 1H), 6.79 (br, 1H), 6.73 (m, 1H), 3.53 (br, 6H), 2.42 (br, 4H) [M+H]+=386.

### EXAMPLE 18

By working as above described in any previous example and by using the suitable starting material as formerly reported, the following compounds of formula (I) of the invention may be thus obtained:
1. 2-(1H-pyrazol-3-yl)-1H-indole
2. 4-fluoro-2-(1H-pyrazol-3-yl)-1H-indole
3. 5-fluoro-2-(1H-pyrazol-3-yl)-1H-indole
4. 6-fluoro-2-(1H-pyrazol-3-yl)-1H-indole
5. 4-chloro-2-(1H-pyrazol-3-yl)-1H-indole
6. 5-chloro-2-(1H-pyrazol-3-yl)-1H-indole
7. 6-chloro-2-(1H-pyrazol-3-yl)-1H-indole
8. 4-bromo-2-(1H-pyrazol-3-yl)-1H-indole
9. 5-bromo-2-(1H-pyrazol-3-yl)-1H-indole
10. 6-bromo-2-(1H-pyrazol-3-yl)-1H-indole
11. 4-cyano-2-(1H-pyrazol-3-yl)-1H-indole
12. 5-cyano-2-(1H-pyrazol-3-yl)-1H-indole
13. 6-cyano-2-(1H-pyrazol-3-yl)-1H-indole
14. 4-nitro-2-(1H-pyrazol-3-yl)-1H-indole
15. 5-nitro-2-(1H-pyrazol-3-yl)-1H-indole
16. 6-nitro-2-(1H-pyrazol-3-yl)-1H-indole
17. 4-methyl-2-(1H-pyrazol-3-yl)-1H-indole
18. 5-methyl-2-(1H-pyrazol-3-yl)-1H-indole
19. 6-methyl-2-(1H-pyrazol-3-yl)-1H-indole
20. 4-trifluoromethyl-2-(1H-pyrazol-3-yl)-1H-indole
21. 5-trifluoromethyl-2-(1H-pyrazol-3-yl)-1H-indole
22. 6-trifluoromethyl-2-(1H-pyrazol-3-yl)-1H-indole
23. 4-methyoxy-2-(1H-pyrazol-3-yl)-1H-indole
24. 5-methyoxy-2-(1H-pyrazol-3-yl)-1H-indole
25. 6-methyoxy-2-(1H-pyrazol-3-yl)-1H-indole
26. 4-hydroxy-2-(1H-pyrazol-3-yl)-1H-indole
27. 5-hydroxy-2-(1H-pyrazol-3-yl)-1H-indole
28. 6-hydroxy-2-(1H-pyrazol-3-yl)-1H-indole
29. 2-(1H-pyrazol-3-yl)-1H-indole-4-carboxylic acid
30. 2-(1H-pyrazol-3-yl)-1H-indole-5-carboxylic acid
31. 2-(1H-pyrazol-3-yl)-1H-indole-6-carboxylic acid
32. methyl 5-(1H-indol-2-yl)-1H-pyrazole-4-carboxylate
33. methyl 5-(1H-indol-2-yl)-1H-pyrazole-5-carboxylate
34. methyl 5-(1H-indol-2-yl)-1H-pyrazole-6-carboxylate
35. ethyl 5-(1H-indol-2-yl)-1H-pyrazole-4-carboxylate
36. ethyl 5-(1H-indol-2-yl)-1H-pyrazole-5-carboxylate
37. ethyl 5-(1H-indol-2-yl)-1H-pyrazole-6-carboxylate
38. i-butyl 5-(1H-indol-2-yl)-1H-pyrazole-4-carboxylate
39. i-butyl 5-(1H-indol-2-yl)-1H-pyrazole-5-carboxylate
40. i-butyl 5-(1H-indol-2-yl)-1H-pyrazole-6-carboxylate
41. 2-(1H-pyrazol-3-yl)-1H-indole-4-carboxamide
42. 2-(1H-pyrazol-3-yl)-1H-indole-5-carboxamide
43. 2-(1H-pyrazol-3-yl)-1H-indole-6-carboxamide
44. N-methyl-2-(1H-pyrazol-3-yl)-1H-indole-4-carboxamide
45. N-methyl-2-(1H-pyrazol-3-yl)-1H-indole-5-carboxamide
46. N-methyl-2-(1H-pyrazol-3-yl)-1H-indole-6-carboxamide
47. N-ethyl-2-(1H-pyrazol-3-yl)-1H-indole-4-carboxamide
48. N-ethyl-2-(1H-pyrazol-3-yl)-1H-indole-5-carboxamide
49. N-ethyl-2-(1H-pyrazol-3-yl)-1H-indole-6-carboxamide
50. N-propyl-2-(1H-pyrazol-3-yl)-1H-indole-4-carboxamide
51. N-propyl-2-(1H-pyrazol-3-yl)-1H-indole-5-carboxamide
52. N-propyl-2-(1H-pyrazol-3-yl)-1H-indole-6-carboxamide
53. N-isopropyl-2-(1H-pyrazol-3-yl)-1H-indole-4-carboxamide
54. N-isopropyl-2-(1H-pyrazol-3-yl)-1H-indole-5-carboxamide
55. N-isopropyl-2-(1H-pyrazol-3-yl)-1H-indole-6-carboxamide
56. N-butyl-2-(1H-pyrazol-3-yl)-1H-indole-4-carboxamide 57. N-butyl-2-(1H-pyrazol-3-yl)-1H-indole-5-carboxamide
58. N-butyl-2-(1H-pyrazol-3-yl)-1H-indole-6-carboxamide
59. N-isobutyl-2-(1H-pyrazol-3-yl)-1H-indole-4-carboxamide
60. N-isobutyl-2-(1H-pyrazol-3-yl)-1H-indole-5-carboxamide
61. N-isobutyl-2-(1H-pyrazol-3-yl)-1H-indole-6-carboxamide
62. N-terbutyl-2-(1H-pyrazol-3-yl)-1H-indole-4-carboxamide
63. N-terbutyl-2-(1H-pyrazol-3-yl)-1H-indole-5-carboxamide
64. N-terbutyl-2-(1H-pyrazol-3-yl)-1H-indole-6-carboxamide
65. N-phenyl-2-(1H-pyrazol-3-yl)-1H-indole-4-carboxamide
66. N-phenyl-2-(1H-pyrazol-3-yl)-1H-indole-5-carboxamide
67. N-phenyl-2-(1H-pyrazol-3-yl)-1H-indole-6-carboxamide
68. N-benzyl-2-(1H-pyrazol-3-yl)-1H-indole-4-carboxamide
69. N-benzyl-2-(1H-pyrazol-3-yl)-1H-indole-5-carboxamide
70. N-benzyl-2-(1H-pyrazol-3-yl)-1H-indole-6-carboxamide
71. N-(2-methoxyethyl)-2-(1H-pyrazol-3-yl)-1H-indole-4-carboxamide
72. N-(2-methoxyethyl)-2-(1H-pyrazol-3-yl)-1H-indole-5-carboxamide
73. N-(2-methoxyethyl)-2-(1H-pyrazol-3-yl)-1H-indole-6-carboxamide
74. N-(3-hydroxypropyl)-2-(1H-pyrazol-3-yl)-1H-indole-4-carboxamide
75. N-(3-hydroxypropyl)-2-(1H-pyrazol-3-yl)-1H-indole-5-carboxamide
76. N-(3-hydroxypropyl)-2-(1H-pyrazol-3-yl)-1H-indole-6-carboxamide
77. 4-(piperidin-1-ylcarbonyl)-2-(1H-pyrazol-3-yl)-1H-indole
78. 5-(piperidin-1-ylcarbonyl)-2-(1H-pyrazol-3-yl)-1H-indole
79. 6-(piperidin-1-ylcarbonyl)-2-(1H-pyrazol-3-yl)-1H-indole
80. N-cyclopentyl-2-(1H-pyrazol-3-yl)-1H-indole-4-carboxamide
81. N-cyclopentyl-2-(1H-pyrazol-3-yl)-1H-indole-5-carboxamide
82. N-cyclopentyl-2-(1H-pyrazol-3-yl)-1H-indole-6-carboxamide
83. 4-[(4-benzylpiperazin-1-yl)carbonyl]-2-(1H-pyrazol-3-yl)-1H-indole
84. 5-[(4-benzylpiperazin-1-yl)carbonyl]-2-(1H-pyrazol-3-yl)-1H-indole
85. 6-[(4-benzylpiperazin-1-yl)carbonyl]-2-(1H-pyrazol-3-yl)-1H-indole
86. 2-(1H-pyrazol-3-yl)-N-(tetrahydrofuran-2-ylmethyl)-1H-indole-4-carboxamide
87. 2-(1H-pyrazol-3-yl)-N-(tetrahydrofuran-2-ylmethyl)-1H-indole-5-carboxamide
88. 2-(1H-pyrazol-3-yl)-N-(tetrahydrofuran-2-ylmethyl)-1H-indole-6-carboxamide
89. 1-{[2-(1H-pyrazol-3-yl)-1H-indol-4-yl]carbonyl}piperidin-4-ol
90. 1-{[2-(1H-pyrazol-3-yl)-1H-indol-5-yl]carbonyl}piperidin-4-ol
91. 1-{[2-(1H-pyrazol-3-yl)-1H-indol-6-yl]carbonyl}piperidin-4-ol
92. N-(3-dimethylamino)propyl-2-(1H-pyrazol-3-yl)-1H-indole-4-carboxamide
93. N-(3-dimethylamino)propyl-2-(1H-pyrazol-3-yl)-1H-indole-5-carboxamide
94. N-(3-dimethylamino)propyl-2-(1H-pyrazol-3-yl)-1H-indole-6-carboxamide
95. 2-(1H-pyrazol-3-yl)-N-(pyridin-2-ylmethyl)-1H-indole-4-carboxamide
96. 2-(1H-pyrazol-3-yl)-N-(pyridin-2-ylmethyl)-1H-indole-5-carboxamide
97. 2-(1H-pyrazol-3-yl)-N-(pyridin-2-ylmethyl)-1H-indole-6-carboxamide
98. 2-(1H-pyrazol-3-yl)-N-(2-pyridin-2-ylethyl)-1H-indole-4-carboxamide
99. 2-(1H-pyrazol-3-yl)-N-(2-pyridin-2-ylethyl)-1H-indole-5-carboxamide
100. 2-(1H-pyrazol-3-yl)-N-(2-pyridin-2-ylethyl)-1H-indole-6-carboxamide
101. N-(4-methoxyphenyl)-2-(1H-pyrazol-3-yl)-1H-indole-4-carboxamide
102. N-(4-methoxyphenyl)-2-(1H-pyrazol-3-yl)-1H-indole-5-carboxamide
103. N-(4-methoxyphenyl)-2-(1H-pyrazol-3-yl)-1H-indole-6-carboxamide
104. N-(4-fluorobenzyl)-2-(1H-pyrazol-3-yl)-1H-indole-4-carboxamide
105. N-(4-fluorobenzyl)-2-(1H-pyrazol-3-yl)-1H-indole-5-carboxamide
106. N-(4-fluorobenzyl)-2-(1H-pyrazol-3-yl)-1H-indole-6-carboxamide
107. N-(2,5-difluorobenzyl)-2-(1H-pyrazol-3-yl)-1H-indole-4-carboxamide
108. N-(2,5-difluorobenzyl)-2-(1H-pyrazol-3-yl)-1H-indole-5-carboxamide
109. N-(2,5-difluorobenzyl)-2-(1H-pyrazol-3-yl)-1H-indole-6-carboxamide
110. N-(2-anilinoethyl)-2-(1H-pyrazol-3-yl)-1H-indole-4-carboxamide
111. N-(2-anilinoethyl)-2-(1H-pyrazol-3-yl)-1H-indole-5-carboxamide
112. N-(2-anilinoethyl)-2-(1H-pyrazol-3-yl)-1H-indole-6-carboxamide
113. N-(5-hydroxy-1H-pyrazol-3-yl)propyl-2-(1H-pyrazol-3-yl)-1H-indole-4-carboxamide
114. N-(5-hydroxy-1H-pyrazol-3-yl)propyl-2-(1H-pyrazol-3-yl)-1H-indole-5-carboxamide
115. N-(5-hydroxy-1H-pyrazol-3-yl)propyl-2-(1H-pyrazol-3-yl)-1H-indole-6-carboxamide
116. N-(3-morpholin-4-ylpropyl)-2-(1H-pyrazol-3-yl)-1H-indole-4-carboxamide
117. N-(3-morpholin-4-ylpropyl)-2-(1H-pyrazol-3-yl)-1H-indole-5-carboxamide
118. N-(3-morpholin-4-ylpropyl)-2-(1H-pyrazol-3-yl)-1H-indole-6-carboxamide
119. N-(2-phenylamino-ethyl)propyl-2-(1H-pyrazol-3-yl)-1H-indole-4-carboxamide
120. N-(2-phenylamino-ethyl)propyl-2-(1H-pyrazol-3-yl)-1H-indole-5-carboxamide
121. N-(2-phenylamino-ethyl)propyl-2-(1H-pyrazol-3-yl)-1H-indole-6-carboxamide
122. N-[2-(1H-imidazol-4-yl)-ethyl]-2-(1H-pyrazol-3-yl)-1H-indole-4-carboxamide
123. N-[2-(1H-imidazol-4-yl)-ethyl]-2-(1H-pyrazol-3-yl)-1H-indole-5-carboxamide
124. N-[2-(1H-imidazol-4-yl)-ethyl]-2-(1H-pyrazol-3-yl)-1H-indole-6-carboxamide
125. N-[3-(1H-imidazol-1-yl)propyl]-2-(1H-pyrazol-3-yl)-1H-indole-4-carboxamide
126. N-[3-(1H-imidazol-1-yl)propyl]-2-(1H-pyrazol-3-yl)-1H-indole-5-carboxamide
127. N-[3-(1H-imidazol-1-yl)propyl]-2-(1H-pyrazol-3-yl)-1H-indole-6-carboxamide
128. N-(4-hydroxy-butyl)-2-(1H-pyrazol-3-yl)-1H-indole-4-carboxamide
129. N-(4-hydroxy-butyl)-2-(1H-pyrazol-3-yl)-1H-indole-5-carboxamide
130. N-(4-hydroxy-butyl)-2-(1H-pyrazol-3-yl)-1H-indole-6-carboxamide
131. N-(2-hydroxymethyl-phenyl)-2-(1H-pyrazol-3-yl)-1H-indole-4-carboxamide
132. N-(2-hydroxymethyl-phenyl)-2-(1H-pyrazol-3-yl)-1H-indole-5-carboxamide
133. N-(2-hydroxymethyl-phenyl)-2-(1H-pyrazol-3-yl)-1H-indole-6-carboxamide
134. N-(4-methoxy-2-methylphenyl)-2-(1H-pyrazol-3-yl)-1H-indole-4-carboxamide
135. N-(4-methoxy-2-methylphenyl)-2-(1H-pyrazol-3-yl)-1H-indole-5-carboxamide
136. N-(4-methoxy-2-methylphenyl)-2-(1H-pyrazol-3-yl)-1H-indole-6-carboxamide
137. N-(2-furylmethyl)-2-(1H-pyrazol-3-yl)-1H-indole-4-carboxamide
138. N-(2-furylmethyl)-2-(1H-pyrazol-3-yl)-1H-indole-5-carboxamide
139. N-(2-furylmethyl)-2-(1H-pyrazol-3-yl)-1H-indole-6-carboxamide
140. N-(pyridin-4-ylmethyl)-2-(1H-pyrazol-3-yl)-1H-indole-4-carboxamide
141. N-(pyridin-4-ylmethyl)-2-(1H-pyrazol-3-yl)-1H-indole-5-carboxamide
142. N-(pyridin-4-ylmethyl)-2-(1H-pyrazol-3-yl)-1H-indole-6-carboxamide
143. N-[(methoxycarbonyl)methyl]-2-(1H-pyrazol-3-yl)-1H-indole-4-carboxamide
144. N-[(methoxycarbonyl)methyl]-2-(1H-pyrazol-3-yl)-1H-indole-5-carboxamide
145. N-[(methoxycarbonyl)methyl]-2-(1H-pyrazol-3-yl)-1H-indole-6-carboxamide
146. N-(ethane-2-sulfonic acid)-2-(1H-pyrazol-3-yl)-1H-indole-4-carboxamide
147. N-(ethane-2-sulfonic acid)-2-(1H-pyrazol-3-yl)-1H-indole-5-carboxamide
148. N-(ethane-2-sulfonic acid)-2-(1H-pyrazol-3-yl)-1H-indole-6-carboxamide
149. 4-[(4-methylpiperazin-1-yl)carbonyl]-2-(1H-pyrazol-3-yl)-1H-indole
150. 5-[(4-methylpiperazin-1-yl)carbonyl]-2-(1H-pyrazol-3-yl)-1H-indole
151. 6-[(4-methylpiperazin-1-yl)carbonyl]-2-(1H-pyrazol-3-yl)-1H-indole
152. 2-(1H-pyrazol-3-yl)-1H-indol-4-amine
153. 2-(1H-pyrazol-3-yl)-1H-indol-5-amine
154. 2-(1H-pyrazol-3-yl)-1H-indol-6-amine
155. N-[2-(1H-pyrazol-3-yl)-1H-indol-4-yl]acetamide
156. N-[2-(1H-pyrazol-3-yl)-1H-indol-5-yl]acetamide
157. N-[2-(1H-pyrazol-3-yl)-1H-indol-6-yl]acetamide
158. N-[2-(1H-pyrazol-4-yl)-1H-indol-4-yl]propanamide
159. N-[2-(1H-pyrazol-4-yl)-1H-indol-5-yl]propanamide
160. N-[2-(1H-pyrazol-4-yl)-1H-indol-6-yl]propanamide
161. 2-methyl-N-[2-(1H-pyrazol-4-yl)-1H-indol-4-yl]propanamide
162. 2-methyl-N-[2-(1H-pyrazol-4-yl)-1H-indol-5-yl]propanamide
163. 2-methyl-N-[2-(1H-pyrazol-4-yl)-1H-indol-6-yl]propanamide
164. N-[2-(1H-pyrazol-4-yl)-1H-indol-4-yl]butanamide
165. N-[2-(1H-pyrazol-4-yl)-1H-indol-5-yl]butanamide
166. N-[2-(1H-pyrazol-4-yl)-1H-indol-6-yl]butanamide
167. N-[2-(1H-pyrazol-4-yl)-1H-indol-4-yl]benzamide
168. N-[2-(1H-pyrazol-4-yl)-1H-indol-5-yl]benzamide
169. N-[2-(1H-pyrazol-4-yl)-1H-indol-6-yl]benzamide
170. N-[2-(1H-pyrazol-4-yl)-1H-indol-4-yl]phenylacetamide
171. N-[2-(1H-pyrazol-4-yl)-1H-indol-5-yl]phenylacetamide
172. N-[2-(1H-pyrazol-4-yl)-1H-indol-6-yl]phenylacetamide
173. 3-methyl-N-[2-(1H-pyrazol-4-yl)-1H-indol-4-yl]butanamide
174. 3-methyl-N-[2-(1H-pyrazol-3-yl)-1H-indol-4-yl]butanamide
175. 3-methyl-N-[2-(1H-pyrazol-6-yl)-1H-indol-4-yl]butanamide
176. N-[2-(1H-pyrazol-4-yl)-1H-indol-5-yl]tiophenecarboxamide
177. N-methyl-N′-[2-(1H-pyrazol-4-yl)-1H-indol-5-yl]urea
178. N-propyl-N′-[2-(1H-pyrazol-4-yl)-1H-indol-5-yl]urea
179. N-benzyl-N′-[2-(1H-pyrazol-4-yl)-1H-indol-5-yl]urea
180. N-phenyl-N′-[2-(1H-pyrazol-4-yl)-1H-indol-5-yl]urea
181. 5-(1H-indol-2-yl)-1H-pyrazol-4-amine
182. 5-(1H-indol-2-yl)-1H-pyrazole-4-carbonitrile
183. 5-(1H-indol-2-yl)-1H-pyrazole-4-carboxylic acid
184. methyl-5-(1H-indol-2-yl)-1H-pyrazole-4-carboxylate
185. ethyl-5-(1H-indol-2-yl)-1H-pyrazole-4-carboxylate
186. propyl-5-(1H-indol-2-yl)-1H-pyrazole-4-carboxylate
187. i-propyl-5-(1H-indol-2-yl)-1H-pyrazole-4-carboxylate
188. butyl-5-(1H-indol-2-yl)-1H-pyrazole-4-carboxylate
189. i-butyl-5-(1H-indol-2-yl)-1H-pyrazole-4-carboxylate
190. 5-(1H-indol-2-yl)-1H-pyrazole-4-carboxamide
191. N-methyl-5-(1H-indol-2-yl)-1H-pyrazole-4-carboxamide
192. N-ethyl-5-(1H-indol-2-yl)-1H-pyrazole-4-carboxamide
193. N-propyl-5-(1H-indol-2-yl)-1H-pyrazole-4-carboxamide
194. N-1-propyl-5-(1H-indol-2-yl)-1H-pyrazole-4-carboxamide
195. N-butyl-5-(1H-indol-2-yl)-1H-pyrazole-4-carboxamide
196. N-1-butyl-5-(1H-indol-2-yl)-1H-pyrazole-4-carboxamide
197. N-benzyl-5-(1H-indol-2-yl)-1H-pyrazole-4-carboxamide
198. N-phenyl-5-(1H-indol-2-yl)-1H-pyrazole-4-carboxamide
199. N-[3-(dimethylamino)propyl]-5-(1H-indol-2-yl)-1H-pyrazole-4-carboxamide.

1. A method for treating diseases caused by and/or associated with an altered protein kinase activity, by administering to a mammal in need thereof an effective amount of a compound of formula (I) 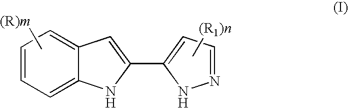
wherein
R is hydrogen, halogen, nitro, cyano, hydroxy, or it is a group optionally further substituted selected from straight or branched C₁-C₆ alkyl or C₁-C₆ alkoxy, C₃-C₆ cycloalkyl, aryl, heterocyclyl, or it is a group —NR′R″, —CONR′R″, —NR′COR″, —COOR′ or —SO₂NR′R″, wherein R′ and R″ are, the same or different and independently in each occasion, a hydrogen atom or a group optionally further substituted selected from straight or branched C₁-C₆ alkyl, C₃-C₆ cycloalkyl, aryl or heterocyclyl; or, taken together with the nitrogen atom to which they are attached, R′ and R″ may form a 5 or 6 membered nitrogen containing heterocycle, optionally comprising one additional heteroatom selected among N, O or S;
R₁ has the meanings above reported to r but other than hydroxy;
m is an integer from 1 to 4;
n is 1 or 2;
and the pharmaceutically acceptable salts thereof.

2. The method according to claim 1 wherein the disease is a cell proliferative disorder selected from the group consisting of cancer, Alzheimer's disease, viral infections, autoimmune diseases and neurodegenerative disorders.

3. The method according to claim 2 wherein the cancer is selected from the group consisting of carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer, and Kaposi's sarcoma.

4. The method according to claim 2 wherein the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis, glomerulonephritis and post-surgical stenosis and restenosis.

5. The method according to claim 1 which provides tumor angiogenesis and metastasis inhibition as well as treatment of organ transplant rejection and host versus graft disease.

6. The method according to claim 1 which provides treatment or prevention of radiotherapy-induced or chemotherapy-induced alopecia.

7. The method according to claim 1 further comprising subjecting the mammal in need thereof to a radiation therapy or chemotherapy regimen in combination with at least one cytostatic or cytotoxic agent.

8. The method according to claim 1 wherein the mammal in need thereof is a human.

9. A method for inhibiting protein kinase activity which comprises contacting the said protein kinase with an effective amount of a compound of formula (I) as defined in claim 1.

10. A compound of formula (I) 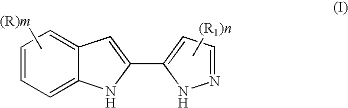
wherein
R is hydrogen, halogen, nitro, cyano, hydroxy, or it is a group optionally further substituted selected from straight or branched C₁-C₆ alkyl or C₁-C₆ alkoxy, C₃-C₆ cycloalkyl, aryl, heterocyclyl, or it is a group —NR′R″, —CONR′R″, —NR′COR″, —COOR′ or
—SO₂NR′R″, wherein R′ and R″ are, the same or different and independently in each occasion, a hydrogen atom or a group optionally further substituted selected from straight or branched C₁-C₆ alkyl, C₃-C₆ cycloalkyl, aryl or heterocyclyl; or, taken together with the nitrogen atom to which they are attached, R′ and R″ may form a 5 or 6 membered nitrogen containing heterocycle, optionally comprising one additional heteroatom selected among N, O or S;
R₁ has the meanings above reported to R but other than hydroxy;
m is an integer from 1 to 4;
n is 1 or 2;
and the pharmaceutically acceptable salts thereof.

11. A compound of formula (I) according to claim 10 wherein R is a hydrogen or halogen atom, R₁ is a hydrogen atom or a group selected from cyano, —COOR′ or —CONR′R″, wherein R′ and R″ are as defined in claim 10, and m and n are both 1.

12. A compound of formula (I) according to claim 10 wherein R is a group —COOR′ or —CONR′R″, wherein R′ and R″ are as defined in claim 10, R₁ is hydrogen, and m and n are both 1.

13. A compound of formula (I) according to claim 10 wherein the optional substituents to any one of the groups R, R₁, R′ and R″ is selected from: halogen, nitro, oxo groups (═O), carboxy, cyano, alkyl, perfluorinated alkyl, hydroxyalkyl, alkenyl, alkynyl, cycloalkyl, aryl, heterocyclyl, amino groups and derivatives thereof such as alkylamino, dialkylamino, cycloalkylamino, arylamino, diarylamino, arylalkylamino, ureido, alkylureido or arylureido; carbonylamino groups and derivatives thereof such as formylamino, alkylcarbonylamino, alkenylcarbonylamino, arylcarbonylamino, alkoxycarbonylamino; hydroxy groups and derivatives thereof such as alkoxy, aryloxy, heterocyclyloxy, alkylcarbonyloxy, arylcarbonyloxy, cycloalkenyloxy or alkylideneaminooxy; carbonyl groups and derivatives thereof such as alkylcarbonyl, arylcarbonyl, alkoxycarbonyl, aryloxycarbonyl, cycloalkyloxycarbonyl, aminocarbonyl, alkylaminocarbonyl, dialkylaminocarbonyl; sulfurated derivatives such as alkylthio, arylthio, alkylsulfonyl, arylsulfonyl, alkylsulfinyl, arylsulfinyl, aryisulfonyloxy, aminosulfonyl, alkylaminosulfonyl or dialkylaminosulfonyl.

14. Any specific compound of formula (I) according to claim 10, optionally in the form of a pharmaceutically acceptable salt, as per the list provided in example 18.

15. A process for preparing the compounds of formula (I) and the pharmaceutically acceptable salts, according to claim 10, which process comprises:
a) coupling, in the presence of a suitable catalyst, the compound of formula (II) with the compound of formula (III) 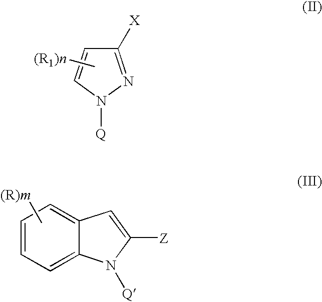
wherein R, R₁, m and n are as defined in claim 10; Q and Q′, the same or different from each other, may represent suitable nitrogen protective groups or polymeric solid supports; X is a halogen atom or a group selected from methylsulfonyloxy, trifluoromethylsulfonyloxy, phenylsulfonyloxy or fluorido-sulphate (—OSO₂F); and Z is selected from halogen, boronic acid, boronate, trialkyl-stannane, trihalostannane, zinc halide, cuprate, alkyldihalo-sylane or a Grignard salt; so as to obtain a compound of formula (IV) 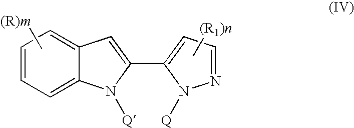
b) optionally converting the compound of formula (IV) into another compound of formula (IV); and
c) deprotecting or cleaving from the resin Q and Q′ the compound of formula (IV), so as to obtain the compound of formula (I) and, whenever desired, converting it into a pharmaceutically acceptable salt thereof.

16. The process of claim 15 wherein the catalyst, in step (a), is selected from tetrakis(triphenylphosphine)palladium, tris(dibenzylideneacetone)dipalladium, palladium chloride, bis(triphenylphosphine)palladium chloride, palladium acetate, nickel chloride, 1,2-bis (diphenylphosphino) ethane nickel chloride, dichlorobis(tributylphosphine)nickel, nickel acetylacetonate and of a suitable ligand such as triphenylphosphine, tri-2-furylphosphine, tributylphosphine, 2-dicyclohexylphosphino-2′-(n,n-dimethylamino)biphenyl, triphenylarsine.

17. The process of claim 15 wherein, within the compounds of formula (II) and (III), X is a iodine atom and Z is a boronic acid [—B(OH)₂] or tributyl stannane.

18. The process of claim 15 wherein Q and Q′, as nitrogen protecting groups, are each independently selected from trityl, trimethylsilylethoxymethyl (SEM), tert-butoxycarbonyl (boc), ethylcarbamate or trichloroethylcarbamate.

19. The process of claim 15 wherein Q and Q′, as suitable polymeric supports, are each independently selected from trityl resin, chloro-trityl resin, methylisocyanate resin, p-nitrophenyl carbonate Wang resin or isocyanate polystyrenic resin.

20. A process for preparing the compounds of formula (I) and the pharmaceutically acceptable salts, according to claim 10, which process comprises:
d) reacting an hydrazine derivative of formula (V) with a pyrazole derivative of formula (VI) 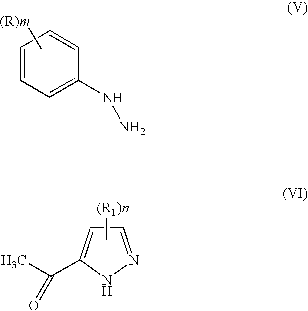
wherein R, R₁, m and n are as defined in claim 10, so as to obtain a compound of formula (VIl) 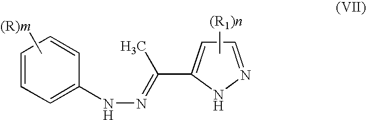
e) reacting the compound of formula (VIl) under acidic conditions and in the presence of a Lewis acid, so as to obtain a compound of formula (I); and,
f) optionally converting it into another compound of formula (I) and/or into a pharmaceutically acceptable salt thereof.

21. The process of claim 21 wherein, in step (e), the Lewis acid is selected from zinc chloride, boron trifluoride, triethylaluminum or trifluoroacetic anhydride.

22. A library of two or more compounds of formula (I) 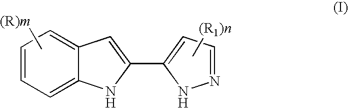
wherein
R is hydrogen, halogen, nitro, cyano, hydroxy, or it is a group optionally further substituted selected from straight or branched C₁-C₆ alkyl or C₁-C₆ alkoxy, C₃-C₆ cycloalkyl, aryl, heterocyclyl, or it is a group —NR′R″, —CONR′R″, —NR′COR″, —COOR′ or —SO₂NR′R″, wherein R′ and R″ are, the same or different and independently in each occasion, a hydrogen atom or a group optionally further substituted selected from straight or branched C₁-C₆ alkyl, C₃-C₆ cycloalkyl, aryl or heterocyclyl; or, taken together with the nitrogen atom to which they are attached, R′ and R″ may form a 5 or 6 membered nitrogen containing heterocycle, optionally comprising one additional heteroatom selected among N, O or S;
R₁ has the meanings above reported to R but other than hydroxy;
m is an integer from 1 to 4;
n is 1 or 2;
and the pharmaceutically acceptable salts thereof.

23. A pharmaceutical composition comprising a therapeutically effective amount of a compound of formula (I), as defined in claim 10, and at least one pharmaceutically acceptable excipient, carrier and/or diluent.

24. A pharmaceutical composition according to claim 23 further comprising one or more chemotherapeutic agents.

25. A product or kit comprising a compound of formula (I) as defined in claim 10 or a pharmaceutical compositions thereof as defined in claim 23, and one or more chemotherapeutic agents, as a combined preparation for simultaneous, separate or sequential use in anticancer therapy.

26. A compound of formula (I) as defined in claim 10 for use as a medicament.

27. Use of a compound of formula (I) as defined in claim 10 in the manufacture of a medicament with antitumor activity.
